(19) United States
(12) Patent Application Publication
(10) Pub. No.: US 2025/0303017 A1
(43) Pub. Date: Oct. 2, 2025

(54) FABRIC COMPOSITIONS COMPRISING COTTON AND HEMP FIBERS, AND METHODS FOR THEIR PREPARATION

(71) Applicant: The United States of America, as represented by the Secretary of Agriculture, Washington, DC (US)

(72) Inventors: DOUG J. HINCHLIFFE, MANDEVILLE, LA (US); JUDSON V. EDWARDS, ABITA SPRINGS, LA (US); NICOLETTE T. PREVOST, PRAIRIEVILLE, LA (US)

(21) Appl. No.: 19/236,334

(22) Filed: Jun. 12, 2025

Related U.S. Application Data
(63) Continuation-in-part of application No. 17/713,871, filed on Apr. 5, 2022.
(60) Provisional application No. 63/171,171, filed on Apr. 6, 2021.

(51) Int. Cl. A61L 15/20; A61L 15/18; D04H 1/02; D04H 1/04; D04H 18/04

(52) U.S. Cl. CPC A61L 15/20; A61L 15/18; D04H 1/02; D04H 1/04; D04H 18/04; D10B 2201/01; D10B 2201/02; D10B 2401/13; D10B 2509/022

(57) ABSTRACT

Disclosed are antimicrobial and/or antiviral fabric compositions of cotton and hemp. In some embodiments, the composition also includes ascorbic acid, citric acid, sodium hypophosphite, and/or a mixture thereof. The ascorbic acid may be covalently attached to at least one cellulosic portion of the fabric composition. Also disclosed are methods for preparing such fabric compositions.

### CROSS-REFERENCE TO RELATED APPLICATIONS

This patent application is a continuation in part (CIP) application of U.S. patent application Ser. No. 17/713,871, filed Apr. 5, 2022, entitled “FABRIC COMPOSITIONS COMPRISING ATTACHED ASCORBIC ACID”, which claims priority to 63/171,171, filed Apr. 6, 2021, entitled “Fabric Compositions Containing One Or More Layers With Ascorbic Acid And/Or Zeolite Or A Zeolite/Pectin Complex Attached to The Fabric Compositions”, each of which is incorporated by reference in its entirety.

### FIELD OF THE INVENTION

The invention relates to antimicrobial and/or antiviral fabric compositions. The invention also relates to antimicrobial and/or antiviral fabric compositions of cotton and hemp further comprising ascorbic acid, citric acid, sodium hypophosphite, or a mixture thereof attached to at least one cellulosic portion of the fabric composition.

### BACKGROUND OF THE INVENTION

The current world-wide textile consumption of both synthetic and natural fibers continues to grow and encompasses a wide range of potential uses for antimicrobial application. However the commercial development of highly effective, low-cost, and environmentally friendly prototypes have had limited success, and with only a few exceptions, commercial approaches to impart antimicrobial activity to textiles have largely been based on the use of synthetic small molecules that are affixed by diffusion or grafting to the fiber by Active Agents (Synthetic Organic Compounds, Metal & Metallic Salts, Bio-based), Application (Medical Textiles, Apparels, Home Textiles), Fabric (Cotton, Polyester, and Polyamide), and Region. Thus, the development of antimicrobial textiles using naturally occurring, non-toxic molecules is of increasing interest. Moreover, this is especially relevant to new applications related to human use for health and hygiene including face masks, wipes, wound dressings, clothes, and medical textiles inclusive of body contacting materials and barrier textiles for medical uses where antibiotic resistance and nosocomial infection is becoming an issue of concern.

The development of robust prolonged field care dressings that have antimicrobial activity accompanying hemostatic function required to treat trauma is obviated in pre-hospital medicine both in civilian and military scenarios. This is especially the case with special forces operations that extend to remote and austere parts of the world where evacuation of casualties is measured in days rather than hours. Microbial growth on textiles that come in contact with the body may double at a rate of 20-30 minutes causing undesirable effects and posing the potential for contamination to the user especially when accessible medical care is inhibited. In addition, the need for an effective hemostatic dressing that has robust antimicrobial activity is also obviated by the virulent activity of Staphylococcus aureus which has evolved mechanisms to gain control over blood coagulation. S. aureus is currently one of the deadliest infectious agents in the developed world, causing intravascular infections such as sepsis and infective endocarditis. Thus, a robust, non-toxic antimicrobial that is effective for prolonged care is ideally suited, and should also: (1) Accelerate clot formation (2) Act as a barrier to microbial contamination and reduce bacterial colony formation; (3) Capable of remaining in place for 72-96 hours without tissue breakdown, reducing the need for frequent dressing changes; (4) Conserve tissue viability by providing a moist environment; and (5) Prevent premature wound closure and formation of fistulae.

Although ascorbic acid has been examined for its antimicrobial activity, it has not been shown to be sufficiently effective when applied alone to impart antibacterial activity in textiles or in solution as would be necessary to prevent contamination when applied to the body. The reason for the low antimicrobial activity has been given in examples from the literature. For example, Vergheses et al. (RJ Verghese, et al., 2017, “Antimicrobial activity of Vitamin C demonstrated on uropathogenic Escherichia coli and Klebsiella pneumoniae,” J. Curr. Res. Sci. Med. 3 (2): 88-93) showed that ascorbic acid alone in solution only partially reduces the microbial growth of Escherichia coli and Klebsiella pneumoniae. Moreover, to date there have been no reports of the use of ascorbic acid as an antibacterial in commercially produced textiles. For this reason, it is clear that despite the low cost, health promoting, and non-toxic nature of ascorbic acid it has eluded commercial textile applications as an antimicrobial. Similarly a non-toxic antiviral fabric for sanitizing surfaces and applicable as a barrier to prevent infectious disease is applicable in wipes, face masks, and hospital barrier fabrics.

Thus, fabric compositions with antimicrobial and/or antiviral properties, and highly effective, low-cost, and environmentally friendly methods for preparing such fabric compositions are urgently needed.

### SUMMARY OF THE INVENTION

Provided herein are affordable, effective, and environmentally-friendly barrier fabrics, and simple and low-cost approaches to preparing such fabric compositions.

In an embodiment, the disclosure relates to antimicrobial and/or antiviral fabric compositions comprising ascorbic acid, citric acid, sodium hypophosphite, or a mixture thereof. The ascorbic acid is covalently attached to at least one cellulosic portion of the fabric composition.

In some embodiments of the disclosure, the antimicrobial and/or antiviral fabric composition is a cloth, a woven fabric, a knitted fabric, a nonwoven fabric, or a final article. In some embodiments of the disclosure, the antimicrobial and/or antiviral fabric composition is a single layered nonwoven fabric or a multilayered nonwoven fabric. In some embodiments of the disclosure, the antimicrobial and/or antiviral fabric composition is a single layered fabric comprising non-scoured, non-bleached greige cotton fibers, bleached cotton fibers, and/or hydrophobic fibers. In some embodiments of the disclosure, the antimicrobial and/or antiviral fabric composition is 100% bleached cotton or greige cotton.

In some embodiments of the disclosure, the antimicrobial and/or antiviral fabric composition is a multi-layered nonwoven fabric composition, comprising at least one inner layer comprising non-scoured, non-bleached greige cotton fibers, and hydrophobic fibers.

In an embodiment the disclosure relates to an article of manufacture prepared with an antimicrobial and/or antiviral fabric composition of the disclosure. In some embodiments of the disclosure the article of manufacture is a protective textile. In some embodiments of the disclosure, the protective textile is a surgical arena fabric, a surgical personnel protective garment, a wound dressing, a non-wound patient dressing, a bandage, a gauze, a packing, a mask, or a cleaning material.

In an embodiment, the disclosure relates to a method for preparing an antimicrobial and/or antiviral fabric composition. The method comprising saturating a fabric composition with citric acid, ascorbic acid, sodium hypophosphite, or a mixture thereof, padding or spraying, and drying the saturated fabric composition at a set first temperature; followed by curing the dried fabric composition at a second, higher temperature than the first temperature.

In one form, the present disclosure provides a single layered nonwoven fabric comprising about 5% by weight to about 95% by weight non-scoured, non-bleached greige cotton fibers, about 0% by weight to about 95% by weight bleached cotton fibers, and about 5% by weight to about 95% by weight hemp fibers, all weight percentages adding up to 100%. In some embodiments, the fabric comprises about 60% by weight non-scoured, non-bleached greige cotton fibers, about 20% by weight bleached cotton fibers, and about 20% by weight hemp fibers. In certain embodiments, the non-scoured, non-bleached greige cotton has a purity level of about 99.9%. In various embodiments, the fabric has a density higher than about 30 g/m². In some embodiments, the fabric has an increased absorption capacity of about 60% to about 70% when compared with a fabric not comprising non-scoured, non-bleached greige cotton fibers. In some embodiments, the fabric further comprises ascorbic acid or sodium ascorbate. In various embodiments, the fabric comprises about 10 mM ascorbic acid or sodium ascorbate. In certain embodiments, the fabric further comprises ascorbic acid, citric acid, sodium hypophosphite, or a mixture thereof.

In some embodiments, the fabric comprises about 60% by weight non-scoured, non-bleached greige cotton fibers, and about 40% by weight unbleached hemp. In various embodiments, the fabric comprises about 55% by weight non-scoured, non-bleached greige cotton fibers, and about 45% by weight bleached hemp. In certain embodiments, the fabric comprises about 60% by weight non-scoured, non-bleached greige cotton fibers, and about 40% by weight unbleached hemp, wherein the fabric has been treated with ascorbic acid. In some embodiments, the fabric comprises about 55% by weight non-scoured, non-bleached greige cotton fibers, and about 45% by weight bleached hemp, wherein the fabric has been treated with ascorbic acid. In some embodiments, the fabrics are treated with 7% (w/v) citric acid, 5% (w/v) L-ascorbic acid, and 4% (w/v) sodium hypophosphite with 0.6% (w/v) 1-hexanol as a wetting agent in ultrapure water using a pad-dry-cure method. In some embodiments, the fabrics are treated with 1% (w/v) L-ascorbic acid with 0.6% (w/v) 1-hexanol as a wetting agent in ultrapure water using a pad-dry-cure method.

In some embodiments, the ascorbic acid is covalently attached to at least one cellulosic portion of the fabric composition. In various embodiments, the fabric composition is a cloth, a woven fabric, a knitted fabric, a nonwoven fabric, or a final article. In certain embodiments, the fabric composition is a nonwoven fabric. In some embodiments, the fabric composition is a woven fabric.

In one form, the present disclosure provides a multilayered nonwoven fabric comprising at least one layer of fabric comprising about 5% by weight to about 95% by weight non-scoured, non-bleached greige cotton fibers, about 0% by weight to about 95% by weight bleached cotton fibers, and about 5% by weight to about 95% by weight hemp fibers, all weight percentages adding up to 100%. In some embodiments, the fabric composition comprises bleached cotton and greige cotton.

In one form, the present disclosure provides an article of manufacture prepared with a fabric composition comprising at least one layer of fabric comprising about 5% by weight to about 95% by weight non-scoured, non-bleached greige cotton fibers, about 0% by weight to about 95% by weight bleached cotton fibers, and about 5% by weight to about 95% by weight hemp fibers, all weight percentages adding up to 100%. In some embodiments, the article of manufacture is a protective textile. In various embodiments, the protective textile is a surgical arena fabric, a surgical personnel protective garment, a wound dressing, a non-wound patient dressing, a bandage, a gauze, a packing, a mask, or a cleaning material.

In one form, the single layered nonwoven fabric is produced by a process comprising: (a) preparing needle punched webs of the fibers, (b) uniformly hydroentangling the webs using a system wherein the system is equipped with one low water pressure jet head that wets the webs on the top face of the webs to form a wetted substrate and wherein the system is equipped with two high water pressure jet heads that subsequently alternatively wets the wetted substrate on either face of the wetted substrate, wherein the system utilizes an about 23 mesh to about 17 mesh screen, and (c) drying the wetted substrate to form the fabric.

In one form, a method is provided for preparing a single layered nonwoven fabric composition, wherein the method comprises saturating a fabric composition with citric acid, ascorbic acid, sodium hypophosphite, or a mixture thereof, padding the saturated fabric composition, drying the padded fabric composition at a first temperature, and curing the dried fabric composition at a second, higher temperature than the first temperature. In some embodiments, the ascorbic acid is covalently attached to at least one cellulosic portion of the fabric composition. In various embodiments, the fabric composition is a cloth, a woven fabric, a knitted fabric, a nonwoven fabric, or a final article. In certain embodiments, the fabric composition is a nonwoven fabric. In some embodiments, the nonwoven fabric is a single layered fabric or a multilayered fabric. In certain embodiments, the fabric composition is a woven fabric. In various embodiments, the fabric composition is 100% bleached cotton or 100% greige cotton. In some embodiments, the fabric composition comprises bleached cotton and greige cotton.

In one form, a multi-layered nonwoven wound dressing is provided, the multi-layered nonwoven wound dressing comprising at least one inner layer containing about 50% by weight to about 95% by weight non-scoured, non-bleached greige cotton fibers and about 5% by weight to about 50% by weight hemp fibers, all weight percentages adding up to 100%, and at least one outer layer containing about 5% by weight to about 95% by weight non-scoured, non-bleached greige cotton fibers, about 0% by weight to about 95% by weight bleached cotton fibers, and about 5% by weight to about 60% by weight hemp fibers, all weight percentages adding up to 100%.

### BRIEF DESCRIPTION OF THE DRAWINGS

FIG. 1 depicts a schematic of a crosslinking reaction of ascorbic acid onto cellulose.

FIG. 2 depicts a schematic of a pad-dry method of the disclosure for ascorbic acid application.

FIG. 3 depicts a schematic of a pad-spray-dry method of the disclosure for the application of zeolite formulation for accelerated hemostatic control. In some embodiments of the pad-spray-dry method, we have separated ascorbic acid application if applied.

FIG. 4 depicts a graph of the hydrogen peroxide produced by fabrics treated in different ways. The Y Axis presents the peroxide concentration in nm per gram of fabric. The X Axis presents the different fabrics: bleached cotton (B8-s2); bleached cotton treated with citric acid and ascorbic acid (B8-s2X1); TACGauze (TGz); TACGauze treated with citric acid and ascorbic acid (TGzX1); BIOGauze (BGz). Hydrogen peroxide content was determined using the ferrous oxidation-xylenol orange assay in quadruplicate, n=4. Error bars represent Standard Error of Mean (SEM). Results were analyzed by ANOVA. Post hoc (Tukey) demonstrated significantly higher levels of H₂O₂ production by TACGauze treated with citric acid and ascorbic acid (TGzX1) or BIOGauze (BGz) versus either bleached cotton (B8-s2) or TACGauze (TGz) alone. * p<0.001 versus bleached cotton. ^{‡} p<0.001 versus TACGauze (TGz).

FIG. 5 depicts a graph of the effects of copper and ascorbic acid on hydrogen peroxide production. The Y Axis presents the peroxide concentration in nanomoles per gram of fabric. The X Axis presents the different fabrics: bleached cotton (B8-s2); 100% cotton woven fabric treated with both copper and ascorbic acid (GCS2); cotton woven fabric treated with ascorbic acid only (GCS1). Hydrogen peroxide content was determined using the ferrous oxidation-xylenol orange assay in quadruplicate, n=4. Error bars represent SEM. Results were analyzed by ANOVA. Post hoc (Tukey) demonstrated significantly higher levels of H₂O₂ production by GCS2 (p<0.001) or by GCS1 (P=0.002) when compared with B8-s2 (made in house (SRRC)).

FIG. 6 depicts a graph of the hydrogen peroxide accumulation over a three-day period. The Y Axis presents the hydrogen peroxide concentration in nanomoles per gram of fabric. The X Axis presents the different days and fabrics: BIOGauze (BGz); TACGauze acid-crosslinked treatment (TGzX1); untreated TACGauze (TGz). Hydrogen peroxide content was determined using the ferrous oxidation-xylenol assay, n=4. Error bars represent SEM. ANOVA (Tukey) revealed that levels of H₂O₂ generated by BGz were significantly higher on Day 2 compared to the other two fabrics. Even though peroxide levels generated with BGz had declined significantly on Day 3, they were significantly higher than either TGzX1 or TGz, whose peroxide levels had fallen to essentially undetectable.

FIG. 7 depicts a graph of the Fourier transform infrared spectra (1850-1500 cm⁻¹) of different fabrics. The Y Axis presents the absorbance (a.u.). The X Axis presents the wavenumber per centimeter (cm⁻¹). The fabrics measured were crosslinked bleached 100% cotton nonwoven fabric with no treatment (B8-s2); treated with 9% citric acid (CA); treated with 9% citric and 5% ascorbic acid (CA+Asc A); treated with 5% ascorbic acid and 3% sodium hypophosphate (Asc A+SHP); treated with 9% citric acid and 3% sodium hypophosphate 3% sodium hypophosphate (CA+SHP); treated with 9% citric acid, 5% ascorbic acid, and 3% sodium hypophosphate (CA+Asc A+SHP).

FIG. 8 depicts a graph of the Fourier transform infrared spectra (1850-1500 cm⁻¹) of crosslinked TACGauze and TACGauze without treatment. The Y Axis presents the absorbance (a.u). The X Axis presents the wave number per centimeter (cm⁻¹). The fabrics measured were crosslinked TACGauze with no treatment (TGz); treated with 9% citric acid (CA); treated with 9% citric acid and 5% ascorbic acid (CA+Asc A); treated with 5% ascorbic acid and 3% sodium hypophosphite (Asc A+SHP); treated with 9% citric acid and 3% sodium hypophosphite (CA+SHP); treated with 9% citric acid, 5% ascorbic acid, and 3% sodium hypophosphite (CA+Asc A+SHP).

FIG. 9 depicts a graph of the hydrogen peroxide produced by fabrics treated in different ways. The Y Axis presents the hydrogen peroxide level (micromolar, μM) of fabric. The X Axis presents the different fabrics: TACGauze (TGz); BIOGauze (BGz); citrate crosslinked TACGauze (CXTGz); TruCotton (TC); unbleached hemp (H), bleached hemp (BH); 60% TruCotton/40% unbleached hemp (60/40 TC/H); 55% TruCotton/45% bleached hemp (55/45 TC/BH); 60% TruCotton/40% unbleached hemp treated with ascorbic acid (60/40 TC/H+AA); and 55% TruCotton/45% bleached hemp treated with ascorbic acid (55/45 TC/BH+AA). Hydrogen peroxide content was determined using the Amplex Red Assay which is performed fluorometrically utilizing Amplex Red reagent (10-acetyl-3,7-dihydroxyphenoxazine) in combination with horseradish peroxidase (HRP). Error bars represent Standard Error of Mean (SEM). Results were analyzed by ANOVA. Post hoc (Tukey) demonstrated significantly higher levels of H₂O₂ production by 60% TruCotton/40% unbleached hemp treated with ascorbic acid (60/40 TC/H+AA) and 55% TruCotton/45% bleached hemp treated with ascorbic acid (55/45 TC/BH+AA) versus TruCotton (TC) alone, unbleached hemp (H) alone, bleached hemp (BH) alone, 60% TruCotton/40% unbleached hemp (60/40 TC/H) alone, and 55% TruCotton/45% bleached hemp (55/45 TC/BH) alone.

### DETAILED DESCRIPTION

The present disclosure relates to affordable, effective, and environmentally-friendly antimicrobial and/or antiviral fabric compositions, and simple and low-cost approaches to preparing such fabric compositions. In some embodiments, the antimicrobial and/or antiviral fabric compositions comprise ascorbic acid, citric acid, sodium hypophosphite, or a mixture thereof covalently attached to at least one cellulosic portion of the fabric composition.

In an embodiment, the disclosure relates to finishing chemistries applied to cellulose-containing fabric compositions to produce antimicrobial and/or antiviral fabric compositions.

Pad/spray dried application and covalent cellulose crosslinking on spunlaced greige cotton nonwovens were found to produce an effective level of antimicrobial activity up to 99.99 percent inhibition. Not wishing to be bound by theory, the associated mechanism of action is thought to be the generation of hydrogen peroxide from the formulated fabrics. Thus, the antimicrobial activity of the ascorbic acid, cotton nonwoven formularies of this study is thought to be based on the classically characterized Fenton reaction. The molecular mechanism is well characterized: in the presence of metal ions as copper or iron, ascorbic acid [(R)-5-[(S)-1,2,-dihydroxyethyl]-3,4-dihydroxyfuran-2 (5H)-one] behaves as a pro-oxidant by cooperatively binding metal ions to form an organometallic bivalent complex, metal-dihydroxyfuranone complex (MDC); and under aerobic conditions MDC binds oxygen (O₂), the core oxygen atoms of hydrogen peroxide, which can then dismutate by way of a protonated reactive oxygen species (ROS) to form hydrogen peroxide as the end product (Zhou, P., et al., 2016, “Generation of hydrogen peroxide and hydroxyl radical resulting from oxygen-dependent oxidation of l-ascorbic acid via copper redox-catalyzed reactions,” RSC Advances 6 (45): 38541-38547). This molecular mechanism initiated in the spunlaced fabric is conceivable both in light of the levels of hydrogen peroxide demonstrated in the treated fabrics and consistent with the presence of transition metal ions previously characterized in these types of cotton fabrics (Edwards, J. V., et al., 2018, “Hydrogen Peroxide Generation of Copper/Ascorbate Formulations on Cotton: Effect on Antibacterial and Fibroblast Activity for Wound Healing Application.” Molecules 23 (9): 2399). An interesting finding of this work is the relative efficacy of the ascorbic acid-nonwoven formulation considering previously published studies that highlight partial antibacterial efficacy. Moreover, the formulated fabrics function effectively at generating hydrogen peroxide levels commensurate with antimicrobial activity for up to two days.

When investigating the ability of cotton to generate hydrogen peroxide the inventors obtained results that are consistent with past reports on the generation of hydrogen peroxide in biological systems (Fry, S. C., 1998, “Oxidative scission of plant cell wall polysaccharides by ascorbate-induced hydroxyl radicals,” Biochem. J. 332 (2): 507-515; Suzuki, N. and Mittler, R., 2012, “Reactive oxygen species-dependent wound responses in animals and plants,” Free Radic. Biol. Med. 53 (12): 2269-2276): 1) cotton can generate hydrogen peroxide at levels that stimulate cell proliferation (Edwards, J. V., et al., 2017, “Induction of Low-Level Hydrogen Peroxide Generation by Unbleached Cotton Nonwovens as Potential Wound Dressing Materials,” J. Funct. Biomater, 8 (1): 9), or 2) addition of ascorbic acid to greige cotton nonwoven fabrics results in robust levels of hydrogen peroxide associated with antibacterial activity (Edwards, J. V., et al., 2018, “Hydrogen Peroxide Generation of Copper/Ascorbate Formulations on Cotton: Effect on Antibacterial and Fibroblast Activity for Wound Healing Application,” Molecules 23 (9): 2399). Thus, the work described here demonstrates the efficacy of formulating unbleached cotton with low add-ons (less than one percent) of ascorbic acid to produce antimicrobial efficacy against both gram-positive and gram-negative bacteria at 99.99 percent inhibition of microbial growth. It is also noteworthy that this antimicrobial design imparts a decidedly ‘green’ motif to the antimicrobial efficacy of the cotton fabric. The mechanism of antibacterial activity is thought to be production of hydrogen peroxide.

Under controlled conditions, the formulation of nonwoven cotton with ascorbic acid results in antibacterial levels of hydrogen peroxide and is also commensurate with antiviral activity. The mechanism is thought to be based on the process outlined above; in the presence of catalytic metals, ascorbate can have pro-oxidant effects, wherein the redox-active metal is reduced by ascorbate, and then reacts with oxygen, producing superoxide. Superoxide dismutates to produce H₂O₂.

Thus, the mode of action and antiviral efficacy has relevance to face mask and barrier textile design. Moreover, numerous questions about the design and efficacy of face mask construction with textiles are receiving increased attention. It is apparent that improvement of face mask efficacy will require highly controlled studies on a wide range of barrier fabrics to optimize efficacy and safety assessments of new designs. For example, numerous studies on the survival of viral particles on different types of surfaces have been performed, with and without disinfectants. However, there are few reports in the literature of studies on active virus titer survival deposited from exhaled and inhaled breath, or a suitable surrogate that simulates respiration into the fabric medium. Thus, evaluating new textile designs that impart virucidal efficacy to cloth face masks in a safe, sustainable, and economical fashion has relevance to the healthcare crisis brought on by SARS-COV-2.

Considering the demonstration of the hydrogen peroxide generation from cotton nonwovens in this study, the levels of hydrogen peroxide reported to be virucidal in the literature are within the range of levels observed in the fabrics of this study. Levels of hydrogen peroxide required to neutralize viral activity as found with SARS-COV-2 have recently been reviewed (Kampf, G., et al., 2020, “Persistence of coronaviruses on inanimate surfaces and their inactivation with biocidal agents,” J. Hosp. Infect. 104 (3): 246-251). Some reports have suggested levels as low as 0.5 percent are adequate, and several reports indicate 1.5 percent. These are levels that are below bacteriostatic hydrogen peroxide. Levels of 3-4 percent hydrogen peroxide are within the bacteriostatic range and are generally accepted to be virucidal. However, viruses would be expected to be less resistant to hydrogen peroxide. Hydrogen peroxide virucidal activity works through the oxidation of lipids and proteins to disrupt the viral replication cycle and prevent host cell entry. In this study the hydrogen peroxide levels are well within this range. Moreover, viruses generally do not have a protective mechanism against hydrogen peroxide as do some bacteria, e.g., catalase neutralization. It is also important to note that some of the fabrics developed in these prototypes contain other constituents that have been reported to be antiviral i.e., pectin and to some extent polyphenolic antioxidants.

In an embodiment, the disclosure relates to antimicrobial and/or antiviral fabric compositions comprising ascorbic acid, citric acid, sodium hypophosphite, or mixtures thereof, where the ascorbic acid is covalently attached to at least one cellulosic portion of the fabric composition using traditional finishing chemistry.

In some embodiments of the disclosure, the fabric composition is a cloth, a woven fabric, a knitted fabric, a nonwoven fabric, or a final article. In some embodiments of the disclosure, the article of manufacture is a medical textile. In some embodiments of the disclosure the medical textile is a surgical arena fabric, a surgical personnel protective garment, a wound dressing, a non-wound patient dressing, a bandage, a gauze, a packing, a mask, or a cleaning material.

In an embodiment, the disclosure relates to antimicrobial and/or antiviral multi-layered fabric compositions containing at least one inner layer and at least one outer layer, where at least one of the inner or outer layers comprises ascorbic acid, citric acid, sodium hypophosphite, or a mixture thereof covalently attached to at least one cellulosic portion of the fabric composition.

In an embodiment of the disclosure, ascorbic acid may be covalently attached to at least one cellulosic portion of a fabric composition. Ascorbic acid may be bound to the fabric compositions using polycarboxylic acids. Examples of polycarboxylic acids that bind ascorbic acid to a fabric composition may be butanecarboxylic acid (BTCA), citric acid (CA), succinic acid (SUA), maleic acid (MLA), or a mixture thereof.

In some embodiments of the disclosure, the cellulosic portion of a fabric composition comprising ascorbic acid, citric acid, sodium hypophosphite, or a mixture thereof covalently attached to at least one cellulosic fiber is from cotton, flax, hemp, jute, ramie, pineapple leaf, wood, bamboo, or abaca.

In an embodiment, the antimicrobial and/or antiviral textile of the disclosure is a felted fabric, a woven fabric, a knitted fabric, a film-based composite, a nonwoven fabric, or a final article. Methods for preparing a fabric composition are known in the art. In some embodiments of the disclosure, a fabric composition comprising ascorbic acid, citric acid, sodium hypophosphite, or a mixture thereof covalently attached to at least one cellulosic fiber is a single layered fabric comprising about 5% by weight to about 95% by weight non-scoured, non-bleached greige cotton fibers; about 0% by weight to about 95% by weight bleached cotton fibers; about 5% by weight to about 60% by weight hydrophobic fibers; all weight percentages adding up to 100%. In some embodiments of the disclosure, the antimicrobial and/or antiviral fabric composition of the disclosure comprises about 60% by weight non-scoured, non-bleached greige cotton fibers, about 20% by weight bleached cotton fibers, and about 20% by weight hydrophobic fibers. In some embodiments of the disclosure, the antimicrobial and/or antiviral fabric composition of the disclosure comprises about 60% by weight non-scoured, non-bleached greige cotton fibers, about 20% by weight bleached cotton fibers, and about 20% by weight hydrophobic fibers. In some embodiments of the disclosure, the antimicrobial and/or antiviral fabric composition of the disclosure comprises about 85% by weight non-scoured, non-bleached greige cotton fibers, and about 15% by weight bleached cotton fibers.

A single layered nonwoven fabric composition may be prepared by any method known in the art. For example, needle punched webs of the different fiber blends may be prepared. Then the needle-punched webs of the different fiber blends may be uniformly hydroentangled using, for example, a Fleissner MiniJet system where the system is equipped with one low water pressure jet head that wets the incoming feed web material on its top face, while two high water pressure jet heads alternatively impact the wetted substrate on either face. For all the fabrics, the low water pressure head may be set to inject the water at about 30 bars, and the two high water pressure heads may be set at about 60 to about 100 bars (e.g., 60 to 100). A 23-mesh screen or lower may be employed to modulate the fabric fenestration. The fabric production speed may be about 5 m per minute. The resulting hydroentangled fabric is dried (e.g., using a meter-wide, gas-fired drum dryer) and may be wound onto a tube (e.g., cardboard) to form a compact fabric roll. One skilled in the art will appreciate, however, that a myriad of other commercial systems having different numbers of jet heads (e.g., many more jet heads) and/or different jet head configurations may be used in lieu of the exemplary Fleissner MiniJet system described above.

A significant amount of the cotton fiber cuticle and primary cell wall components are retained during hydroentanglement, but it is expected that increasing pressure removes more of the non-cellulosic fiber components. The non-cellulosic components can potentially detach or be removed from the fiber matrix due to the force of the water jets that creates an entangled fiber network and also exerts pressure, shear and friction on the outer cuticle layer of the fiber to an extent that this hydrophobic component (contains waxes) of the fiber begins to loosen or even detach from the secondary cell wall of the fiber. Not wishing to be bound by theory, it is hypothesized that these cotton and/or hemp fiber components, which are partially retained from the hydroentanglement process, also play a role in the antimicrobial/antiviral activity of the fabric compositions (e.g., wound dressing material). Moreover, the hydrophobicity afforded by the waxes creates a negatively charged surface that would be resistant to microorganisms and viral particles.

In some embodiments of the disclosure there are multi-layered fabric compositions which contain two or three layers or more. In some embodiments of the disclosure the multilayered fabric composition comprises at least one nonwoven layer. The at least one nonwoven layer in the multi-layered fabric composition of the disclosure may be an inner layer or an outer layer. Methods of preparation of such multi-layers nonwoven fabric compositions are well known in the art.

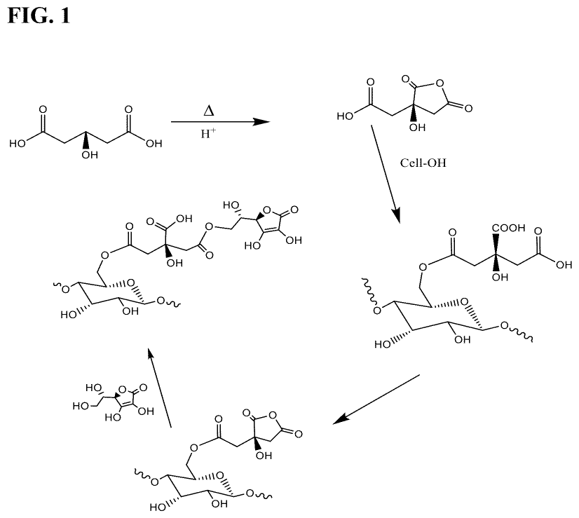

The fabric compositions of the disclosure may be comprised of ascorbic acid treatments alone or as a substrate for delivery of the zeolite formulations to give both antimicrobial and hemostatic fabric compositions singularly and in combination. The ascorbic acid treatment with an add-on to the fabric of less than 1% or from 1-50 percent is antibacterial at 99.99 percent against both gram negative and gram-positive bacteria. Depicted on FIG. 1 is the chemistry of covalently attaching ascorbic acid to cellulose by way of polycarboxylic acid.

The antibacterial and/or antiviral fabric compositions of the disclosure may be a yarn, a thread, a twine, a rope, a cloth, a woven fabric, a knitted fabric, a film-based composite, a nonwoven fabric, or a final article. In some embodiments of the disclosure, the fabric compositions comprising ascorbic acid, citric acid, sodium hypophosphite, or a mixture thereof covalently attached to at least one cellulosic portion of the fabric are a medical textile such as a surgical arena fabric, a surgical personnel protective garment, a wound dressing, a non-wound patient dressing, a bandage, a gauze, a packing, a mask, or a cleaning material.

In some embodiment, a fabric composition of this disclosure may be a nonwoven fabric, which contains greige cotton and hemp along with other hydrophilic and hydrophobic fibers, the combination of which can produce rapid clotting as defined by both thromboelastography (TEG) and in vitro clotting experiments.

Greige cotton refers to unfinished cotton fibers that have not been scoured and bleached. The potential to use greige cotton in nonwoven absorbent products has received increased attention based on innovations in cotton cleaning and processes for nonwovens that open and expose the hydrophilic cellulosic component of greige cotton fiber to water absorption.

Hydrophobic fibers include TruCotton which is a non-scoured, non-bleached 100% natural greige cotton fiber which has been carefully mechanically cleaned to unprecedented levels. Since the cotton fiber has not been chemically altered, the natural waxes and oils remain on the fiber which allows for exceptional processing characteristics in any textile or nonwoven staple fiber manufacturing scheme. TruCotton fiber is naturally hydrophobic, which sets it apart from any cotton fiber previously used for consumer goods. TruCotton is 99.99% pure, meaning that 99.99% of foreign matter (e.g., cotton harvest contaminants in the form of cotton leaves, stems, and bracts; in other words, foreign matter includes anything in the way of trash that is carried over from the field to the ginning process) has been removed. The staple fiber length is about 19 to about 30 mm, hydrophobicity reflected in the water contact angle which is 140.9°+5.3, and has a denier (micronaire) of about 3.5 to about 5.5 (e.g., 3.5 to 5.5; preferably about 4.0 to about 5.5 (e.g., 4.0 to 5.5)). Other hydrophobic fibers similar to TruCotton may be used.

Antibacterial and H₂O₂ Activity of Cotton/Hemp Fabrics

In some embodiments, the present disclosure has to do with the production and application of cotton and hemp based fabrics that produce hydrogen peroxide and exert antibacterial activity on both gram negative and positive bacteria. Hydrogen peroxide levels are produced at varying levels that can be construed as applicable to wound healing by way of cell proliferation (low level hydrogen peroxide) or by way of higher levels that are correlated with antibacterial activity. In such an embodiment, the present disclosure solves a variety of problems having to do with hemp and cotton utilization by way of giving rise to commercial wipes, wound dressings, or geo-textile or crop protectants. By way of example, without limitation, additional end-uses of the fabrics disclosed herein may include: Per- and polyfluoroalkyl substances (PFAS) omniphobic alternative finishes that are biobased and biodegradable; heat/flame resistant textiles; textiles with enhanced abrasion resistance for industrial applications; and augmentation of existing antimicrobial properties imparted by metal nanotechnology.

In one form, the present disclosure provides a single layered nonwoven fabric comprising about 5% by weight to about 95% by weight non-scoured, non-bleached greige cotton fibers, about 0% by weight to about 95% by weight bleached cotton fibers, and about 5% by weight to about 95% by weight hemp fibers, all weight percentages adding up to 100%. The fabric may, for example, comprise about 60% by weight non-scoured, non-bleached greige cotton fibers, about 20% by weight bleached cotton fibers, and about 20% by weight hemp fibers. In some embodiments, the non-scoured, non-bleached greige cotton may have a purity level of about 99.9%. In some embodiments, the fabric may have a density higher than about 30 g/m2. In some embodiments, the fabric may have an increased absorption capacity of about 60% to about 70% when compared with a fabric not comprising non-scoured, non-bleached greige cotton fibers. In some embodiments, the fabric further may have ascorbic acid or sodium ascorbate. The fabric may, for example, have about 10 mM ascorbic acid or sodium ascorbate. In some embodiments, the fabric further may include ascorbic acid, citric acid, sodium hypophosphite, or a mixture thereof.

In some embodiments, the fabric may have about 60% by weight non-scoured, non-bleached greige cotton fibers, and about 40% by weight unbleached hemp. In other embodiments, the fabric may have about 55% by weight non-scoured, non-bleached greige cotton fibers, and about 45% by weight bleached hemp. In some embodiments, the fabric comprises about 60% by weight non-scoured, non-bleached greige cotton fibers, and about 40% by weight unbleached hemp, wherein the fabric has been treated with ascorbic acid. In other embodiments, the fabric comprises about 55% by weight non-scoured, non-bleached greige cotton fibers, and about 45% by weight bleached hemp, wherein the fabric has been treated with ascorbic acid. In some embodiments, the fabrics are treated with 7% (w/v) citric acid, 5% (w/v) L-ascorbic acid, 4% (w/v) sodium hypophosphite with 0.6% (w/v) 1-hexanol as a wetting agent in ultrapure water using a pad-dry-cure method as follows: dry at 100° C. for 2 minutes and cure at 160° C. for 5 minutes. In some embodiments, the fabrics are treated with 1% (w/v) L-ascorbic acid with 0.6% (w/v) 1-hexanol as a wetting agent in ultrapure water using a pad-dry-cure method as follows: dry 100° C. for 2 minutes and cure at 160° C. for 1 minute. Alternatively, ascorbic acid treatment can also be performed as follows: dry/cure (i.e., both dry and cure) at 160° C. for 2 minutes. For both procedures, pressure is adjusted for wet pickup of 120-150%.

In some embodiments, the ascorbic acid may be covalently attached to at least one cellulosic portion of the fabric composition. In some embodiments, the fabric composition may be a cloth, a woven fabric, a knitted fabric, a nonwoven fabric, or a final article. The fabric composition may, in a first embodiment, be a nonwoven fabric. The fabric composition may, in a second embodiment, be a woven fabric.

In one form, the present disclosure provides a multilayered nonwoven fabric comprising at least one layer of fabric comprising about 5% by weight to about 95% by weight non-scoured, non-bleached greige cotton fibers, about 0% by weight to about 95% by weight bleached cotton fibers, and about 5% by weight to about 95% by weight hemp fibers, all weight percentages adding up to 100%. In some embodiments, the fabric composition comprises bleached cotton and greige cotton.

In one form, the present disclosure provides an article of manufacture prepared with a fabric composition comprising at least one layer of fabric comprising about 5% by weight to about 95% by weight non-scoured, non-bleached greige cotton fibers, about 0% by weight to about 95% by weight bleached cotton fibers, and about 5% by weight to about 95% by weight hemp fibers, all weight percentages adding up to 100%. In some embodiments, the article of manufacture is a protective textile. In various embodiments, the protective textile is a surgical arena fabric, a surgical personnel protective garment, a wound dressing, a non-wound patient dressing, a bandage, a gauze, a packing, a mask, or a cleaning material.

In one form, the single layered nonwoven fabric is produced by a process comprising: (a) preparing needle punched webs of the fibers, (b) uniformly hydroentangling the webs using a system wherein the system is equipped with one low water pressure jet head that wets the webs on the top face of the webs to form a wetted substrate and wherein the system is equipped with two high water pressure jet heads that subsequently alternatively wets the wetted substrate on either face of the wetted substrate, wherein the system utilizes an about 23 mesh to about 17 mesh screen, and (c) drying the wetted substrate to form the fabric.

In one form, a method for preparing a single layered nonwoven fabric composition, wherein the method comprises saturating a fabric composition with citric acid, ascorbic acid, sodium hypophosphite, or a mixture thereof, padding the saturated fabric composition, drying the padded fabric composition at a first temperature, and curing the dried fabric composition at a second, higher temperature than the first temperature. In some embodiments, the ascorbic acid may be covalently attached to at least one cellulosic portion of the fabric composition. In some embodiments, the fabric composition may be a cloth, a woven fabric, a knitted fabric, a nonwoven fabric, or a final article. The fabric composition may, in a first embodiment, be a nonwoven fabric. The fabric composition may, in a second embodiment, be a woven fabric. In some embodiments, the nonwoven fabric may be a single layered fabric or a multilayered fabric. In some embodiments, the fabric composition may be 100% bleached cotton or 100% greige cotton, along with hemp. In some embodiments, the fabric composition comprises bleached cotton and greige cotton.

In one form, a multi-layered nonwoven wound dressing is provided, the multi-layered nonwoven wound dressing comprising at least one inner layer containing about 50% by weight to about 95% by weight non-scoured, non-bleached greige cotton fibers and about 5% by weight to about 50% by weight hemp fibers, all weight percentages adding up to 100%, and at least one outer layer containing about 5% by weight to about 95% by weight non-scoured, non-bleached greige cotton fibers, about 0% by weight to about 95% by weight bleached cotton fibers, and about 5% by weight to about 60% by weight hemp fibers, all weight percentages adding up to 100%.

Other components (e.g., other hydrophilic or hydrophobic components) known in the art may be added to the fabric compositions of the disclosure provided they do not substantially interfere with the intended activity and efficacy of the fabric compositions; whether or not a compound interferes with activity and/or efficacy can be determined, for example, by the procedures utilized below. Hydrophilic fibers include, for example, bleached and scoured cotton, polyurethane, rayon, spandex, polyacrylate, flax, hemp, ramie, bamboo, alginate, chitosan, hyaluronan, regenerated cellulose, N-acetylglucosamine, and carboxymethylcellulose. Hydrophobic fibers include, for example, polyolefin, polyester, polyacrylate, wool, glass filament, collagen, polypropylene, and nylon.

The terms “fabric” and “textile” are used interchangeably herein, and refer to a cloth, a woven fabric, a knitted fabric, a nonwoven fabric, or an article of manufacture. In some embodiments, the article of manufacture may be a protective textile such as a surgical arena fabric, a surgical personnel protective garment, a wound dressing, a non-wound patient dressing, a bandage, a gauze, a packing, a mask, or a cleaning material.

The terms “wound dressing”, “wound plaster”, “wound bandage” or “wound covering” are used interchangeably herein, and describe dressings for topical application onto external wounds, in order to prevent penetration of foreign bodies into the wound and to absorb blood and wound secretions. Wound dressings are not limited to a particular size or shape. A wound dressing may be a single layer fabric composition, or may be a multi-layered fabric composition. For example, a wound dressing may be in the form of a trilayer fabric composition, comprising two outer layers and an inner layer. A multilayer wound dressing fabric composition has been described herein as comprising first, second and third layers, although it may comprise further layers, such as fourth, fifth, sixth, seventh, eighth, ninth, tenth layers, or more. The further layers may comprise any of the features referred to herein in relation to the inner and outer layers. This also applies to fabric compositions in general.

A fabric composition of the disclosure may contain at least one active substance which is biostatic, biocidal, antimicrobial, disinfecting, inflammation-inhibiting, analgesic, styptic, wound healing-promoting, or a mixture thereof. Thus, the fabric composition of the disclosure may be treated with at least one biostatic, biocidal, antimicrobial, disinfecting, analgesic, inflammation-inhibiting, styptic, wound healing, or a mixture thereof. An antimicrobial substance may be antibacterial, antifungal, or antiviral. Equally it can be provided that the fabric composition of the disclosure contains at least one active substance that is antimicrobial, disinfecting, analgesic, inflammation-inhibiting, styptic, wound healing-promoting, or a mixture thereof. In this connection, it has been found particularly advantageous if the active substance has a biocidal or biostatic action, in particular a bactericidal, bacteriostatic, a fungicidal, fungistatic, virucidal, virostatic action. Specific examples shown in this work include the attachment of ascorbic acid to greige cotton fibers and nonwovens from less than one percent to 50 percent by weight. Ascorbic acid when combined with greige cotton produces both an antimicrobial and antiviral activity of 99.99%.

Antimicrobial Formularies and Their Activity: Formulations are categorized by the function they impart to the dressing,—antimicrobial activity only, or, both, antimicrobial and hemostatic control. Similar reagents are used though their purpose may differ in certain formulations.

Antimicrobial Activities: Table 2 summarizes the add-ons of TACGauze treatments with ascorbic acid and sodium ascorbate. As seen the percent add-on at padding pressures from 5 to 30 psi were typically less than one percent. Given this result in the formulary the antimicrobial results as judged by the AATCC 100 test show that 99.99 percent antimicrobial activity results from treatment with ascorbic acid. Whereas slightly reduced activity was observed with sodium ascorbate at a padding pressure of 5 psi and at 30 psi, no antibacterial activity is observed.

FIG. 1 shows the chemistry of covalently attaching ascorbic acid to the cellulose in cotton by way of a polycarboxylic acid. Table 3 summarizes the add-ons of crosslinking ascorbic acid. Percent add-ons ranged from less than one percent to greater than nine percent based on the formulations which included various types of approaches to acid-catalyzed ascorbic acid crosslinking.

Table 8 shows the results of antiviral activity against TACGauze and BIOGauze. It is notable that BIOGauze gave 99.99% reduction in viral load after one hour contact with the surface of the fabric.

Antimicrobial Formulary Hemostatic Activity: Table 4 and Table 5 summarize TEG clotting results of the ascorbic acid crosslinked fabrics in combination with one and ten percent zeolite, and BIOGauze formulated with sodium zeolite and pectin, which demonstrated favorable clotting commensurate with hemorrhage control activity as shown in Table 5.

Table 4 summarizes some of the TEG clotting results of the ascorbic acid crosslinked fabrics in combination with one and ten percent zeolite. However, this approach appears not to favor improved clotting profiles. On the other hand, BIOGauze formulated with sodium zeolite and pectin demonstrated favorable clotting commensurate with hemorrhage control activity as shown in Table 5.

Table 10 summarizes antimicrobial results using the AATCC 100 test method, which is the industry standard for antimicrobial fabric performance in the United States. For this analysis, Gram-positive Staphylococcus aureus American Type Culture Collection (ATCC) 6538 (S. aureus) and Gram-negative Klebsiella pneumoniae ATCC 4352 (K. pneumoniae) were selected for use in this assay as they represent organisms that are commonly found to colonize and infect wounds. As shown in Table 1, TACGauze (TGz) had no effect on either Gram positive or negative organism whereas BGz, without additions, and both samples of greige cotton with ascorbic acid finishes resulted in a 99.99 percent reduction in gram positive and gram-negative bacterial growth.

Table 9 defines TACGauze, 100% cotton woven and nonwoven fabrics and their defined treatments with ascorbic acid. Initially TACGauze (TGz) was treated with ascorbic acid resulting in an antibacterial prototype termed BIOGauze (BGz). Table 10 shows that BGz, a formulation of a greige cotton-based spunlaced nonwoven with ascorbic acid, produces 99.99 percent antibacterial activity.

The permissive host cell for MS2 bacteriophage is Escherichia coli. As seen in Table 11, the study showed that BGz reduced viral load by 99.99 percent after one hour compared with the control and TGz, BGz precursor, at time zero. As seen in Table 12, BGz provided a 90% reduction of Human coronavirus, Strain 229E, with no reduction seen with TGz or commercial N-95 mask sample at a time point of six hours. It is also important to note that the 90% inhibition observed with the Coronavirus is expected at the minimum since the results were blurred by host cell modifications that occurred during the virus assay, i.e., lifting of E. coli cells in the petri dish.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

“Optional” or “optionally” means that the subsequently described event or circumstance may or may not occur, and that the description includes instances in which the event or circumstance occurs and instances where it does not. For example, the phrase “optionally comprising an antimicrobial agent” means that the fabric composition of the invention may or may not contain an antimicrobial agent and that this description includes fabric compositions that contain and do not contain an antimicrobial agent. Also, by example, the phrase “optionally adding an antimicrobial agent” means that the method may or may not involve adding an antimicrobial agent and that this description includes methods that involve and do not involve adding an antimicrobial agent.

Other compounds (e.g., antimicrobial agent) may be added to the fabric compositions of the invention provided they do not substantially interfere with the intended activity and efficacy of the fabric compositions; whether or not a compound interferes with activity and/or efficacy can be determined, for example, by the procedures utilized below.

By the term “effective amount” of a compound or property as provided herein is meant such amount is capable of performing the function of the compound or property for which an effective amount is expressed. As will be pointed out below, the exact amount required will vary from process to process, depending on recognized variables such as the compounds employed, and the processing conditions observed. Thus, it is not possible to specify an exact “effective amount.” However, an appropriate effective amount may be determined by one of ordinary skill in the art using only routine experimentation.

The amounts, percentages, and ranges disclosed herein are not meant to be limiting, and increments between the recited amounts, percentages, and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions (e.g., reaction time, temperature), percentages and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as much as 10% to a reference quantity, level, value, or amount.

As used herein, the term “about” is defined as plus or minus ten percent of a recited value. For example, about 1.0 g means 0.9 g to 1.1 g.

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

Regarding double entries in the tables, there is either some slight variation in the formulation or the add-ons vary. Thus, duplicate entries in terms of samples tested.

All of the references cited herein, including patents and patent application Publications, are incorporated by reference in their entirety. Also incorporated by reference in their entirety are the following references: Wagner, W., et al., J. Surgical Res., 66:100-108 (1996); U.S. Pat. Nos. 6,809,231; 9,474,827; 9,463,119; U.S. Patent Application Publication Number 20170128270; U.S. Patent Application Publication Number 20190380878; U.S. patent application Ser. No. 16/110,169; U.S. Pat. No. 11,246,756; U.S. patent application Ser. No. 17/713,871; and U.S. Patent Application Publication Number 20220323635.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein).

The invention illustratively disclosed herein suitably may be practiced in the absence of any element (e.g., method (or process) steps or composition components) which is not specifically disclosed herein. Thus, the specification includes disclosure by silence (“Negative Limitations In Patent Claims,” AIPLA Quarterly Journal, Tom Brody, 41 (1): 46-47 (2013): “ . . . . Written support for a negative limitation may also be argued through the absence of the excluded element in the specification, known as disclosure by silence . . . . Silence in the specification may be used to establish written description support for a negative limitation. As an example, in Ex parte Lin [No. 2009-0486, at 2, 6 (B.P.A.I. May 7, 2009)] the negative limitation was added by amendment . . . . In other words, the inventor argued an example that passively complied with the requirements of the negative limitation . . . was sufficient to provide support . . . . This case shows that written description support for a negative limitation can be found by one or more disclosures of an embodiment that obeys what is required by the negative limitation . . . .”

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

The following examples are intended only to further illustrate embodiments of the disclosure and are not intended to limit the scope of the invention as defined by the claims.

### EXAMPLES

Having now generally described this invention, the same will be better understood by reference to certain specific examples, which are included herein only to further illustrate embodiments of the disclosure and are not intended to limit the scope of the invention as defined by the claims.

### Example 1

### Materials and Methods

Disclosed in this example are the materials and methods used herein to develop fabric compositions comprising ascorbic acid attached to at least one of its fibers.

Preparation of Single Layered Nonwoven Material. Hydroentanglement of fibrous webs into nonwoven fabric structures: A commercially available bale of pre-cleaned greige cotton was acquired from T. J. Beall, LLC (now Wildwood Cotton Technologies, Greenwood, Mississippi, USA). Polypropylene fibers were acquired commercially. A bleached version of TruCotton raw cotton was also acquired from T. J. Beall/Wildwood Cotton Technologies. The needle punched webs of the different fiber blends were uniformly hydroentangled using a Fleissner MiniJet system. The system was equipped with one low water pressure jet head that wets the incoming feed web material on its top face while two high water pressure jet heads alternatively impact the wetted substrate on either face. For all the fabrics, the low water pressure head was set to wet the fabric at 30 bars of water pressure and the two high water pressure heads were set at either 60, 80, or 100 bars. The fabric production speed was 5 meters per minute. The resulting hydro-entangled fabric was dried using a meter-wide, gas-fired, through-Drum Dryer and wound onto a cardboard tube to form a compact fabric roll. The hydroentangling line utilizes municipal water that is passed through a reverse osmosis filter that is set to give a water hardness of 70 to 110 PPM.

Use in face mask: Material (greige cotton woven or nonwoven) was cut for front and back of mask. Antiviral nonwoven (AVN) was adhered to the inside facing, or was directly adhered to the outside as a covering. Thus, availing a protective antiviral barrier to entry or exit of active virus. The mask facings were sewn a quarter inch in. A face material piece was placed over each AVN before sewing. Pins embedded in fabric showed how and where to sew. Ancillary pieces such as nose guard and elastic mask-to-ear anchors were incorporated to complete the basic mask construct.

Fabric Treatment. Materials and Reagents: All other chemicals and fabrics were from existing supply/inventory. The l-ascorbic acid, and calcium chloride (CaCl₂)) were purchased from Sigma Aldrich (now Millipore Sigma; Burlington, Massachusetts, USA). Sodium hypophosphite monohydrate (NaH₂PO₂·H₂O) was from JT Baker (Phillipsburg, New Jersey, USA). Ultrapure water (1862), water was obtained using a Milli-Q water purification system (Millipore-Sigma), and was used as solvent. The fabrics used were as follows: TACGauze (TGz) from H&H Medical Corporation (Williamsburg, Virginia, USA), a blend of 50% greige cotton/30% bleached cotton/20% polypropylene; Fine Mesh Gauze (FMGz), 100% bleached cotton (#4-2915 inside roll 36″ 50-yard roll from DeRoyal Industries (Powell, Tennessee, USA); Hydroentangled nonwoven fabric (NW85), 85% TruCotton (greige cotton) and 15% bleached cotton (TruCotton) produced at Southern Regional Research center (SRRC) (New Orleans, Louisiana, USA); and 100% cotton plain weave printcloth (GC), a quilting fabric by Dover Hill in “Burlap Pewrer” purchased from Benatrex. For spray application of formulation, an Aldrich-flask type thin layer chromatography (TLC) sprayer was used.

Treatments done for Antimicrobial Activity only were: (1) 10 mM Ascorbic acid alone; (2) 0.95% (w/w) or ˜50 mM ascorbic acid and 0.6% (w/w) 1-hexanol (BIOGauze); (3) Citric acid (CA) only; (4) Ascorbic acid (Asc A) only; (5) CA+Asc A; (6) CA+sodium hypophosphite hydrate (SHP); (7) CA+Asc A+SHP; and (8) Asc A+SHP.

Treatments done for Antimicrobial Activity and Hemostatic Control were: (1) Varying weight percent citric acid, ascorbic acid, sodium hypophosphite and powdered Y zeolite; (2) Varying weight percent citric acid, ascorbic acid, sodium hypophosphite, pectin and powdered Y zeolite; (3) Varying weight percent citric acid, ascorbic acid, sodium hypophosphite, pectin, CaCl₂) and powdered Y zeolite. BIOGauze (or pretreatment of greige cotton/cotton blend with 0.95% (w/w) ascorbic acid & 0.6% (w/w) 1-hexanol) then treated with either: (1) Y Zeolite alone in water; (2) varying weight percent of pectin and powdered Y zeolite; (3) 1% CaCl₂) and varying percent powdered Y zeolite; (4) varying weight percent of pectin, CaCl₂) and powdered Y zeolite; (5) 0.5% Pectin, 0.5M sodium carbonate, 1% CaCl₂); and (6) 0.5% Pectin, 0.5M sodium carbonate, 1% CaCl₂) and varying percent of powdered NaY zeolite. Formulations were made with NaY or NH₄Y as the zeolite.

### Example 2

### Application of Ascorbic Acid and Zeolites

Two methods were originally used in the application of ascorbic acid and/or zeolite to the fabrics. Each method could have a variation of either one or two steps.

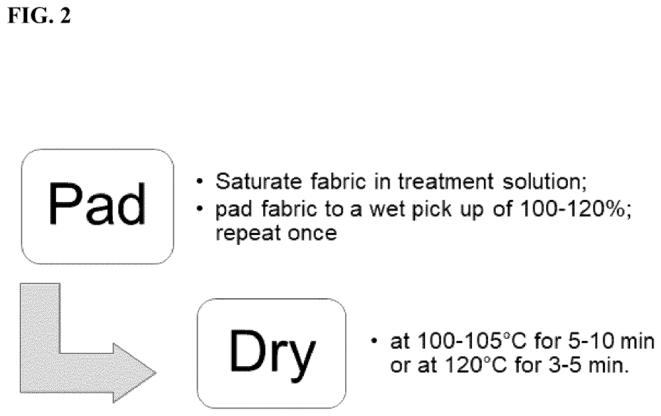

Application Method 1: Pad-Dry, a schematic diagram of which is shown in FIG. 2. To treat the fabrics, fabric swatches were submersed and saturated in a solution volume of 20 times the weight of fabric. The saturated swatches were padded with a CALLINGER hand-cranked wringer. Padding was repeated and the wet padded weight of the swatch was recorded. The swatches were dried on a screen or metal frame in a force draft oven of either 100° C. to 105° C. for 5 to 10 minutes or 120° C. for 3 to 5 minutes without tension. Swatches were not rinsed. The formulation swatches equilibrated overnight before weight measurement. Swatches were equilibrated overnight at ambient conditions and weighed.

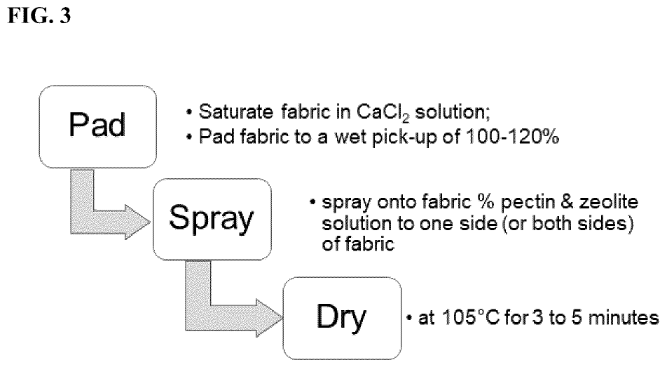

Application Method 2: Pad-Spray-Dry, a schematic diagram of which is shown in FIG. 3. Fabric swatches were saturated with a weight percent CaCl₂) solution volume 20 times its weight, and were padded to remove excess solution. Then, swatches were clipped to a frame and sprayed with a percent solution of ascorbic acid or pectin and zeolite previously vortexed using an aspirator (glass nozzle and flask attached to house air). Acrylic acid at low aqueous concentrations was used as an adherent. Spraying application was in a sweeping motion and applied to single-side of two-sided fabric surface. The fabric formulation was dried for 3 minutes at 105° C. Without rinsing, fabrics were equilibrated overnight to room atmosphere and humidity and then weighed. In the embodiment of the pad-spray-dry method shown in FIG. 3, the zeolite formulation (i.e., % pectin and zeolite) is applied for accelerated hemostatic control. In some embodiments of the pad-spray-dry method, the application of ascorbic acid, if applied, may be separated from the application of the zeolite formulation.

### Example 3

### Antimicrobial Activities

Antimicrobial activities of greige cotton-containing-materials treated with ascorbic acid were tested.

BIOGauze: Ascorbic acid and TACGauze: Ascorbic Acid Antimicrobial Finish. Purpose: Treatment of greige cotton-containing material (60/20/20, Greige Cotton/Bleached Cotton/Polypropylene) to impart antimicrobial activity. The padding/ovens used in processing were used to simulate commercial processing. Formulations: Solution 1:10 mM Ascorbic acid, 1.76 g deionized water (designated house tap), 1 L; Solution 2:10 mM Sodium ascorbate, 1.98 g, deionized water (designated house tap), 1 L. Machine Settings: Matthis padder: speed 2; three padding pressures 5 psi, 15 psi, and 30 psi; continuous drying oven (Matthis): temperature 85° C.; web run: 0.047 m/min=9 minutes 56 seconds; samples were 25 inches length by 4 inches wide (62.5 cm×10.2 cm). Data shown in the tables below.

BIOGauze: Pilot process run: The fabric formulation consisted of 0.95% (w/wt) of Ascorbic acid and 0.6% (w/w) 1-hexanol added as wetting agent. The process run treated the fabric using the following conditions: fabric roll was padded with a wet pick up of 121%; processed for 20 seconds through ovens with oven temperature (zone 1/zone 2) 315° F./320° F. (157° C./160° C.) then IR web temperature (zone1/zone2): 240° F./320° F. (115° C./160° C.) at a line speed of 37 feet per minute (fpm). The targeted exit temperature was 320° F./160° C.

Selected fabrics were submitted to Situbiosciences (Wheeling, Illinois, USA) for fabric testing, AATCC TM 100 test method designed to measure the antimicrobial properties of textile or absorbent material incubated with selected microorganisms. These samples were tested against K. pneumoniae (4352) and S. aureus (6538). At time 0, the bacterial levels were 7.6×10⁴ CFU/mL for K pneumoniae and 5.3×10⁵ CFU/mL for S. aureus. The levels of K pneumoniae and S. aureus at 24 hours are noted in Table 1 below. Sample #4 was retested and no change in result, ineffective against these bacteria.

TABLE 1
AATCC 100 TM RESULTS

| | | | K pneumoniae | S. aureus |
|---|---|---|---|---|
| # | Sample ID | Sample Description | % Reduction after 24 hours | |
| 1 | 062118-1 | 5 psi 10 mM | 99.99 | 99.99 |
| | | Ascorbic acid | | |
| 2 | 062118-5 | 30 psi 10 mM | 99.35 | 99.99 |
| | | Ascorbic acid | | |
| 3 | 062118-7 | 5 psi 10 mM | 99.95 | 95.09 |
| | | Sodium Ascorbate | | |
| 4 | 062118-12 | 30 psi 10 mM | 0 | 0 |
| | | sodium Ascorbate | | |
| 5 | 062618-1A-B | 10 mM Ascorbic | 99.99 | 99.99 |
| | | acid(NTP Ctrl) | | |
| 6 | 062118-3 | Untreated | 0 | 0 |
| | | TACGauze Ctrl | | |
| 7 | Situ lab Ctrl | K. pneumoniae/ | 8.2E6 CFU/mL | 3.0E6 CFU/mL |
| | | S. aureus | | |

Antimicrobial Activities: Table 2 summarizes the add-ons of TACGauze treatments with ascorbic acid and sodium ascorbate. As seen, the percent add-on at padding pressures from 5 to 30 psi were typically less than one percent. Given this result in the formulary, the antimicrobial results as judged by the AATCC 100 test show that 99.99 percent antimicrobial activity results from treatment with ascorbic acid. Whereas slightly reduced activity was observed with sodium ascorbate at a padding pressure of 5 psi and at 30 psi, no antibacterial activity was observed.

TABLE 2
Percent add-on of TACGauze first antimicrobial treatment

| Sample ID | Sample Description | Wet pickup % | % Add-On |
|---|---|---|---|
| 062118-1 | 5 psi 10 mM Ascorbic acid | 211.31 | 0.22 |
| 062118-2 | 5 psi 10 mM Ascorbic acid | 215.65 | −0.23 |
| 062118-3 | 15 psi 10 mM Ascorbic acid | 217.51 | 0.44 |
| 062118-4 | 15 psi 10 mM Ascorbic acid | 215.21 | 0.00 |
| 062118-5 | 30 psi 10 mM Ascorbic acid | 322.32 | 0.42 |
| 062118-6 | 30 psi 10 mM Ascorbic acid | 155.30 | 0.21 |
| 062118-7 | 5 psi 10 mM Sodium Ascorbate | 449.69 | 1.25 |
| 062118-8 | 5 psi 10 mM Sodium Ascorbate | 440.12 | 1.23 |
| 062118-9 | 15 psi 10 mM Sodium Ascorbate | 209.68 | 0.42 |
| 062118-10 | 15 psi 10 mM Sodium Ascorbate | 211.06 | 0.43 |
| 062118-11 | 30 psi 10 mM Sodium Ascorbate | 213.27 | 0.41 |
| 062118-12 | 30 psi 10 mM Sodium Ascorbate | 211.51 | 0.63 |

### Example 4

Antiviral Properties of Formulations with Crosslinked Ascorbic Acid

The ability to crosslink ascorbic acid to cotton fabrics was investigated using different procedures.

Two different process conditions were used for crosslinking Ascorbic Acid to Cotton-Fabrics: The processes utilized differing reagent concentrations, cure temperatures, and duration. Solutions 20× the weight of fabric were made to treat the swatches along with controls. Process condition I: Six 40 mL solutions were made from 20% (w/v) stock solutions of the main reagents with resulting solution concentrations of 9% (w/v) citric acid (CA), 5% (w/v) ascorbic acid (Asc A), and 3% (w/v) sodium hypophosphite (SHP) (NaH₂PO₂·H₂O) to treat the swatches. To each solution, 0.6% (w/v) of TRITON X-100 non-ionic surfactant was added as wetting agent. They were as follows: (1) Citric acid (CA) only; (2) Ascorbic acid (Asc A) only; (3) CA+Asc A; (4) CA+sodium hypophosphite hydrate (SHP); (5) CA+Asc A+SHP; and (6) Asc A+SHP. Swatches were saturated and padded with Matthis padder (set at 30 lbs psi) adjusted to give about 100-120% wet pickup and dried at 95° C. for 3 minutes. Fabrics were heated using Mathis oven for 5 minutes at 165° C. to cure. Process condition II: 7% CA+4.8% SHP only; 7% CA+4.8% SHP+1% Asc A. Swatches were saturated, padded and dried for 3 minutes at 95° C. and cured for 3 minutes at 160° C. All swatches were then rinsed with deionized water. Excess water was removed by padder and dried in oven at 100° C. for 3 minutes. They were weighed after equilibrating overnight.

Table 3 summarizes the add-ons of crosslinking ascorbic acid. Percent add-ons ranged from less than one percent to greater than nine percent based on the formulations which included various types of approaches to acid-catalyzed ascorbic acid crosslinking.

TABLE 3
Percent add-on data of cotton fabrics*
crosslinked with citric acid, ascorbic acid

| | | % Wet | Final |
|---|---|---|---|
| Sample ID | Sample Description | Pickup | % Add-On |
| 020720-1 | TACGz 9% Citric acid alone | 114.16 | 3.13 |
| 020620-2 | TACGz 5% Ascorbic acid alone | 126.90 | 0.45 |
| 020620-3 | TACGz 9% CA + 5% Asc A | 119.86 | 1.30 |
| 020620-4 | TACGz 9% CA + 3% SHP | 126.40 | 6.82 |
| 020620-5 | TACGz 9% CA + 5% Asc A + 3% SHP | 122.15 | 7.16 |
| 020620-6 | TACGz 5% Asc A + 3% SHP | 105.75 | 0.39 |
| 020720-2 | B8S-2 9% Citric acid alone | 108.38 | 4.68 |
| 020620-8 | B8S-2 5% Ascorbic acid alone | 108.05 | 1.56 |
| 020620-9 | B8S-2 9% CA + 5% Asc A | 118.54 | 2.79 |
| 020620-10 | B8S-2 9% CA + 3%SHP | 109.84 | 7.79 |
| 020620-11 | B8S-2 9% CA + 5% Asc A + 3% SHP | 117.31 | 9.43 |
| 020620-12 | B8S-2 5% AscA + 3%SHP | 102.10 | 1.91 |
| 020720-3 | FMGz 9% Citric acid alone | 124.31 | 3.82 |
| 020620-14 | FMGz 5% Ascorbic acid alone | 120.41 | 1.13 |
| 020620-15 | FMGz 9% CA + 5% AscA | 134.92 | 1.63 |
| 020620-16 | FMGz 9% CA + 3% SHP | 122.35 | 6.53 |
| 020620-17 | FMGz 9% CA + 5% Asc A + 3% SHP | 112.84 | 8.54 |
| 020620-18 | FMGz 5% Asc A + 3%SHP | 112.52 | 0.76 |
| 020620-1 | TACGz 9% Citric acid alone * | 300.75 | 5.06 |
| 020620-7 | B8S-2 9% Citric acid alone * | 229.71 | 6.62 |
| 020620-13 | FMGz 9% Citric acid alone * | 162.56 | 4.68 |

CA = citric acid,
Asc A = Ascorbic acid,
SHP = sodium hypophosphite monohydrate
(NaH₂PO₂•H₂O),
* = 2-hexanol was added as wetting agent at 0.6 % (w/v).

Antimicrobial and hemostatic. Crosslinking: ascorbic acid and zeolite formulations: Four 40 mL solutions were made to treat the swatches. They were as follows: (1) 7% (w/v) citric acid (CA) and 4.8% (w/v) sodium hypophosphite monohydrate (SHP) (NaH₂PO₂·H₂O); (2) 7% (w/v) CA and 4.8% (w/v) NaH₂PO₂·H₂O, and 1% (w/v) ascorbic acid (Asc.A.), ˜54 mM; (3) 7% (w/v) CA and 4.8% (w/v) NaH₂PO₂·H₂O, 1% (w/v) Asc. A. and 1% (w/v) Sodium Y zeolite (NaY); (4) 7% (w/v) CA and 4.8% (w/v) NaH₂PO₂·H₂O, 1% (w/v) Asc. A. and 10% (w/v) NaY. Swatches were saturated, padded and dried for 3 minutes at 95° C.; then, they were cured for 2 minutes at 160° C. All swatches were then rinsed with deionized water. They were padded to remove excess water and dried in oven at 100° C. for 3 minutes. They were weighed after equilibrating overnight.

Antimicrobial Formulary Hemostatic Activity: Table 4 and Table 5 summarize TEG clotting results of the ascorbic acid crosslinked fabrics in combination with one and ten percent zeolite, and BIOGauze formulated with sodium zeolite and pectin. Table 4 summarizes some of the TEG clotting results of the ascorbic acid-crosslinked fabrics in combination with one and ten percent zeolite. However, this approach appears not to favor improved clotting profiles. On the other hand, as shown in Table 5, BIOGauze, TACGauze treated with ascorbic acid and XX formulated with sodium zeolite and pectin demonstrated favorable clotting commensurate with hemorrhage control activity.

TABLE 4
Various Cotton Fabric swatches crosslinked with Ascorbic acid and zeolite

| | | % Add | R | | K | |
|---|---|---|---|---|---|---|
| Sample ID | Description | on | (min) | σ | (min) | σ |
| 062419-1 | TACGz CA + NaH₂PO₂ | 5.71 | | | | |
| 062419-2 | TACGz CA + SHP + Asc A | 4.93 | | | | |
| 062419-3 | TACGz CA + SHP + Asc A + 1% NaY | 6.93 | 8.8 | | 7.4 | |
| 062419-4 | TACGz CA + SHP + Asc A + 10% NaY | 8.86 | 8.7 | | 5.1 | |
| 062419-5 | B9S-2 CA + SHP | 5.50 | | | | |
| 062419-6 | B9S-2 CA + SHP + Asc A | 6.29 | | | | |
| 062419-7 | B9S-2 CA + SHP + Asc A + 1% NaY | 7.61 | | | | |
| 062419-8 | B9S-2 CA + SHP + Asc A + 10% NaY | 4.43 | 5.9 | 0.1 | 5.1 | 0.5 |
| 062419-9 | B8S-2 CA + SHP | 7.41 | | | | |
| 062419-10 | B8S-2 CA + SHP + Asc A | 6.74 | | | | |
| 062419-11 | B8S-2 CA + SHP + Asc A + 1% NaY | 6.97 | | | | |
| 062419-12 | B8S-2 CA + SHP + Asc A + 10% NaY | 3.75 | 5.8 | 0 | 6.4 | 0.7 |
| 062419-13 | Fine Mesh Gz CA + SHP | 4.79 | | | | |
| 062419-14 | FMGz CA + SHP + Asc A | 3.14 | | | | |
| 062419-15 | FMGz CA + SHP + Asc A + 1% NaY | 3.80 | | | | |
| 062419-16 | FMGz CA + SHP + Asc A + 10% NaY | 4.17 | 8.8 | 0.9 | 7.2 | 0.9 |
| 062419-17 | FMGz CA + SHP + Asc A | 5.81 | | | | |
| 062419-18 | FMGz, CA + SHP | 5.74 | | | | |
| 062019-11 | BIOGz CA + SHP | 4.01 | | | | |
| | Blood | — | 12.6 | 0.4 | 5.4 | 0.5 |
| | Procoagulant | — | 3.4 | | 2.5 | |

CA = citric acid, Asc A = Ascorbic acid, SHP = sodium hypophosphite monohydrate (NaH₂PO₂•H₂O); TACGz = 60/20/20 greige cotton/polypropylene/bleach cotton blend hydroentangled nonwoven; B9S-2 = 100% greige cotton Hydroentangled nonwoven; B8S-2 = 100% bleached hydroentangled nonwoven; FMGz = 100% bleached fine mesh gauze(DeRoyal), BIOGz = previously treated TACGauze with ascorbic acid formulation.

TABLE 5
Pad-dry application of zeolite and pectin on BIOGauze

| | | % Add- | R | | K | |
|---|---|---|---|---|---|---|
| Sample ID | Sample Description | On | (min) | σ | (min) | σ |
| 050119-1 | 10% NaY only | 36.1 | 5.0 | 0.4 | 3.3 | 0.3 |
| 050119-2 | 0.25% Pectin only | 0.5 | 9.9 | 1.2 | 7.0 | 1.1 |
| 050119-3 | 0.5% Pectin only | 1.6 | 10.2 | 1.9 | 6.2 | 2.0 |
| 050119-4 | 0.25% PEC + 10% NaY | 27.4 | 5.4 | 0.7 | 3.7 | 1.4 |
| 050119-5 | 0.5% PEC + 10% NaY | 35.2 | 5.4 | 0.2 | 2.8 | 0.4 |
| | Blood | — | 13.6 | 0.9 | 7.6 | 1.4 |
| | Procoagulant | — | 4.3 | 0.3 | 3.3 | 0.8 |
| 062019-1 | 10% NaY only | 38.7 | 4.4 | 0.1 | 1.6 | 0.1 |
| 062019-3 | 1% CaCl₂ + 10% NaY | 53.5 | 4.6 | | 1.7 | |
| 062019-5 | 0.25% PEC + 10% NaY | 41.2 | 4.6 | 1.0 | 2.8 | 2.2 |
| 062019-6 | 0.25% PEC + 10% NaY | 36.8 | | | | |
| 062019-7 | 0.5% PEC + 10% NaY | 27.6 | 5.7 | 0.3 | 2.4 | 0.3 |
| 062019-9 | 0.5M Na₂CO₃ + 1% CaCl₂ + 10% NaY | 37.0 | | | | |
| | Blood (bovine) | n/a | 19.6 | 2.2 | 8.7 | 1.7 |
| | Procoagulant | | 4.3 | 0.4 | 1.8 | 0.1 |

Abbreviations:
Na₂CO₃ = sodium carbonate,
PEC = pectin,
NaY = sodium
Y zeolite,
CaCl₂ = calcium chloride

As shown in Table 6 the combination of sodium zeolite with pectin is somewhat comparable to employing alginate. When sodium carbonate and calcium chloride were employed in the formulation with pectin the time to clot formation was generally within the range expected for a procoagulant but time to fibrin formation was somewhat slower.

TABLE 6
Pad-dry* on BIOGauze

| | | % Add- | R | | K | |
|---|---|---|---|---|---|---|
| Sample ID | Sample Description | On | (min) | σ | (min) | σ |
| 050219-1 | 1% CaCl₂ + 10% NaY only | 44.5 | 4.4 | 0.5 | 2.0 | 0.1 |
| 050219-2 | 0.5% Na Alginate, 0.5M Na₂CO₃ + 1% | 16.4 | 12.3 | | 6.2 | |
| | CaCl₂ only | | | | | |
| 050219-3 | 0.5% Na Alginate, 0.5M Na₂CO₃ + 1% | 28.9 | 6.9 | 0.4 | 2.7 | 1.4 |
| | CaCl₂ + 10% NaY | | | | | |
| 050219-4 | 0.5% Pectin, 0.5M Na₂CO₃ + 1% | 17.8 | 10.7 | | 3.5 | |
| | CaCl₂ only | | | | | |
| 050219-5 | 0.5% Pectin, 0.5M Na₂CO₃ + 1% | 28.4 | 6.4 | 0.1 | 2.4 | 0.2 |
| | CaCl₂ + 10% NaY | | | | | |
| 050219-6 | 0.5M Na₂CO₃, 1% CaCl₂, 10% NaY | 33.6 | 4.8 | 0.1 | 1.8 | 0.1 |
| 050219-7 | 0.5M NaOH, 1% CaCl₂, 10% NaY | 32.6 | 6.5 | 0.6 | 3.0 | 0.4 |
| 050219-8 | 10% NaY in 1 mM SDS | 17.8 | 6.2 | 0.4 | 2.0 | 0 |
| 050219-9 | 0.1M CaO, 1 mM SDS, 10% NaY | 34.5 | 5.8 | 0.1 | 2.0 | 0.3 |
| 050219-10 | 0.1M CaO, 1 mM SDS, only | 7.9 | 10.7 | 0.3 | 4.6 | 0.3 |
| | Blood | — | 12.7 | 2.2 | 4.9 | 0.4 |
| | Procoagulant | — | 3.3 | 0.0 | 1.4 | 0.1 |

*Stepwise reagent addition to padbath. Soak, pad then dry.
Abbreviations:
Na₂CO₃ = sodium carbonate,
CaO = Calcium oxide,
SDS = Sodium dodecyl sulfate,
Na Alginate = alginic acid, sodium salt

Table 7 shows that the use of calcium oxide did not improve on this trend. The use of spray applications to TACGauze showed comparable clotting times commensurate with procoagulant hemorrhage control.

TABLE 7
Comparison of spray to pad-dry method
TEG data of TACGauze and BIOGauze*

| | | % Add- | R | | K | |
|---|---|---|---|---|---|---|
| Sample ID | Sample Description | On | (min) | σ | (min) | σ |
| | TACGz | | 9.7 | 0.8 | 5.3 | 0.7 |
| | | | 10.2 | | 6.5 | |
| | | | 9.6 | 0.8 | 4.8 | 1.0 |
| | BIOGz | | 8.3 | 1.6 | 2.7 | 0.6 |
| | | | 10.6 | 0.4 | 8.7 | 3.0 |
| 082019-1 | TACGz 0.5% PEC, | 40.4 | 5.2 | 0.0 | 1.4 | 0.1 |
| | 2% CaCl2, 5% NaY | | 6.7 | 0.1 | 3.4 | 0.5 |
| | | | 4.9 | 0.1 | 1.5 | 0.2 |
| 022819-10 | TACGz 0.25% PEC, | 43.4 | 6.5 | 0.5 | 2.0 | 0.5 |
| | 10% NaY | | 6.6 | 0.6 | 3.4 | 0.1 |
| 050119-4 | BIOGz 0.25% PEC, | 27.4 | 5.4 | 0.2 | 1.0 | 0.1 |
| | 10% NaY | | 6.0 | 0.0 | 3.3 | 0.6 |
| 050219-1 | BIOGz 1% CaCl₂, | 44.5 | 5.1 | 0.0 | 1.4 | 0.2 |
| | 10% NaY | | 5.6 | 0.4 | 3.6 | 0.1 |
| | | | 4.2 | 0.1 | 1.2 | 0.3 |
| 081919-1 | TACGz 1% CaCl₂, | 24.5 | 6.1 | | 1.3 | |
| | 2% PEC, 10% NaY (S) | | 5.1 | 0.2 | 1.5 | 0.3 |
| 022819-5 | TACGz 0.5% PEC + | 45.7 | 6.1 | 1.0 | 1.5 | 0.1 |
| | 10% NaY | | | | | |
| 050119-5 | BIOGz 0.5% PEC + | 35.2 | 5.8 | 0.2 | 1.4 | 0.1 |
| | 10% NaY | | | | | |
| 031319-8 | TACGz 1% CaCl₂, | 28.8 | 5.1 | 0.2 | 1.5 | 0.5 |
| | 10% NaY | | | | | |
| | bovine blood | | 12.9 | 0.4 | 2.9 | 0.2 |
| | | | 18.2 | 0.9 | 9.2 | 2.4 |
| | | | 13.4 | 0.2 | 4.0 | 0.4 |
| | Procoagulant | | 3.8 | 0.1 | 0.8 | 0.0 |
| | | | 3.8 | | 2.3 | |
| | | | 3.0 | 0.1 | 0.8 | 0.1 |

*In instances where the measurements were done during a different date/run, numbers for each run are on the same line.
Abbreviations:
TACGz = TACGauze,
BIOGz = BIOGauze,
PEC = pectin,
NaY = sodium Y zeolite,
CaCl₂ = calcium chloride

Antiviral Assay of TACGauze and BIOGauze: Antiviral tests were conducted at MicroChem Laboratory (Austin, Texas, USA). The test microorganism(s) selected for this test: MS2 Bacteriophage (MS2), ATCC 15597-B1. This virus is a non-enveloped positive-stranded RNA virus of the bacteriophage family Leviviridae. Bacterial cells are the hosts for bacteriophages, and E. coli 15597 serves this purpose for MS2 bacteriophage. Its small size, icosohedral structure, and environmental resistance has made MS2 ideal for use as a surrogate virus (particularly in place of picornaviruses such as poliovirus and human norovirus) in water quality and disinfectant studies. Permissive Host Cell System for MS2: Escherichia coli, 15597.

Fabric articles were stacked with 4-6 swatches per stack, or a sufficient number of swatches to absorb approximately 1.0 mL of inoculum. The microorganisms were prepared by initiating a test culture in appropriate growth broth and incubating for about 24 hours. The inoculum was prepared from the 24-hour incubated culture by diluting to the specified concentration. The culture diluent that may be used in this method ranges from full strength nutritive broth to reverse osmosis/deionized water or anything in between. For hydrophobic articles, a surfactant such as Triton X-100 non-ionic surfactant was added to the culture diluent. Once prepared, the inoculum was plated to confirm the starting concentration. A milliliter of inoculum was added to each stack of fabric articles or an appropriate volume as designated by the nature of the article or specified by the Study Sponsor.

A subset of control articles was harvested following inoculation to determine the starting concentration on the fabric. Following inoculation, the test articles and parallel control articles were incubated in closed sealed Petri dishes inside a sealed plastic bag/container under incubation conditions optimal for the microorganism, typically 36° C. Upon completion of the contact time, dictated by the length of incubation, the test and control articles were harvested. Carriers were harvested by aseptically folding stacks and placing them into neutralizer media, then vortex mixing for about 1 to 2 minutes. The neutralizer was then plated using standard dilution and plating techniques.

Enumeration plates are incubated at the appropriate conditions for 24 to 48 hours. Sterility controls, including test and control articles, were performed on each day of testing. The microorganisms used in the study were checked for purity. Neutralization verifications were performed if requested by the Study Sponsor prior to testing initiation.

The assays were replicated 3 times, and Table 8 lists the test microorganism (Microorganism), the contact time (Time), the Carrier, the Average PFU/carrier; and the percent reduction and the Log 10 reduction. Both reductions were compared to the control at time Zero. MS2Bacteriophage ATCC 15597-B1. The limit of detection for this assay is 1.00E+01 PFU/carrier and is reported as <1.00E+01 PFU/carrier in the table below.

TABLE 8
MS2Bacteriophage ATCC 15597-B1 detection

| | | | Average | | |
|---|---|---|---|---|---|
| | | | PFU/ | Percent | Log10 |
| Microorganism | Time | Carrier | Carrier | Reduction | Reduction |
| MS2 | Zero | Control | 2.23E+06 | N/A | N/A |
| Bacteriophage | Six | Control | 2.00E+05 | N/A | N/A |
| | hours | | | | |
| ATCC | Six | TacGauze | <1.67E+01 | >99.99925% | >5.13 |
| 15597-B1 | hours | | | | |
| | Six | BioGauze | <1.00E+01 | >99.99955% | >5.35 |
| | hours | | | | |
| | One | BioGauze | 2.13E+02 | 99.99% | 3.84 |
| | hour | | | | |
| | One | TacGauze | 9.67E+05 | 34.09% | 0.18 |
| | hour | | | | |

The data presented in this Example shows BIOGauze, even after only one hour presented with anti-bacterial properties, and that the antibacterial properties improved for TACGauze and BIOGauze with exposure time.

### Example 5

### Ascorbic Acid Encapsulation

Encapsulated ascorbic acid was prepared to attach to textiles converting them to antimicrobial and/or antiviral textiles.

All chemicals and fabrics were from existing supply/inventory. L-Ascorbic acid, pectin, (from citrus peel) and calcium chloride dihydrate were from Sigma Aldrich (now Millipore Sigma). Zeolite sodium Y faujasite (NaY) from Zeolyst. Ultrapure water (1852) was used as solvent.

The Buchi encapsulator B-390 was used to make microbeads/capsules. We used air-assisted nozzle of size 750 μm which uses a prilling by vibration technique. You can make liquid core beads or wet microcapsules that you can dry. The electrode and frequency along with air pressure are variables to adjust to produce the beads.

1% (w/v) pectin and 1% (w/v) ascorbic acid in water. 0.5% pectin with 1% ascorbic acid was used first but did not form microbeads in the 2% (w/v) calcium chloride casting solution. The casting solution which was stirred as the solution was dropped into it. A 2% (w/v) calcium chloride in 50% aqueous ethanol was used to encapsulate ascorbic acid in pectin. The settings were about 75 mbar for air pressure, 700 Hz frequency and 800V for electrode. Solution 2: 1% (w/v) pectin and 5% (w/v) NaY zeolite and the casting solution was 4% CaCl₂. The air pressure was 100-300 mbar, and 900 Hz and 900V for frequency and electrode.

Both solutions formed beads, which can be attached to dressings through pad dry cure or spray approaches to yield antimicrobial fabric.

### Example 6

### Antiviral Testing

This Example was performed to determine whether the commercially based hemostatic fabric, TACGauze, could be modified by traditional finishing chemistry treatments to create a fabric that retained its hemostatic function but also possessed significant antibacterial and antiviral properties.

Selected fabrics were submitted to Microchem Laboratory (Round Rock, Texas, USA) for antiviral fabric testing, AATCC-100 test method for antibacterial finishes on textile materials modified for viruses. The screening assay used Bacteriophage (MS2) ATCC15597-B1 with Escherichia coli 15597 as the permissive host cell system. The assay was repeated with two controls and the test microorganism was Human coronavirus, strain 229E, ATCC VR-740 with host cell MRC-5 (ATCC CCL-171), human lung fibroblast cell line. The procedures (4.2.5.1 and 4.2.5.2) used at Microchem Laboratory are summarized below.

Test samples/articles were cut into 4.8 cm diameter circles (5/stack). Control fabric was steam sterilized. The inoculum was prepared from a frozen stock culture by diluting to the specified concentration using phosphate buffered saline. Once prepared, the inoculum was plated to confirm the starting concentration. A milliliter of inoculum was added to each stack of fabric articles. A subset of control articles was harvested following inoculation to determine the starting concentration on the fabric. Following inoculation, the test articles and parallel control articles were incubated in closed sealed Petri dishes inside a sealed plastic bag/container under incubation conditions optimal for the microorganism, typically 36° C. Upon completion of the contact time(s), dictated by the length of incubation, the test and control articles were harvested. Carriers were harvested by aseptically folding stacks and placing them into neutralizer media, D/E Broth, then mixed for about 1 to 2 minutes by vortex. The neutralizer was then plated using standard dilution and plating techniques. Enumeration plates are incubated at the appropriate conditions for 12 to 24 hours. Sterility controls, including test and control articles, were performed on each day of testing. The microorganisms used in the study were checked for purity.

The stock virus was thawed and was not supplemented with an organic soil load. From the test samples, 1-inch squares were cut and were placed into sterile plastic Petri dishes. To begin contact time, a 0.100 to 0.200 ml volume of virus was inoculated onto each sample surface while ensuring equal distribution for swatches. The Petri dish was then covered for the duration of the contact time. At the completion of the contact time, each sample was aseptically transferred to a sterile conical tube containing an appropriate volume of neutralization media and vortexed. After vortexing, a 0.1 ml aliquot was used in a series of 10-fold dilutions of appropriate test medium. Each dilution was inoculated into the appropriate host cells in quadruplicate. The log and percent reduction in viral titer were calculated using the test and recovery control titer results. The control swatch was tested in the same manner as test sample swatches. For the cytotoxicity control, the test procedure was followed with the exception that the aliquot of test medium was used to inoculate the control in lieu of virus. To confirm test substance neutralization, an aliquot of the dilutions prepared in the cytotoxicity control was inoculated into the host cells. The cells were incubated at the appropriate test conditions for approximately 7 days and were microscopically observed for test virus cytotoxic effect and the virus titer.

Table 9 defines TACGauze, the fabric identification, and its treatments/methods with ascorbic acid. The table also identifies 100% cotton woven and nonwoven fabrics and their defined treatments. Initially TACGauze (TGz) was treated with ascorbic acid resulting in an antibacterial prototype termed BIOGauze (BGz). Table 10 shows that BGz, a formulation of a greige cotton-based spunlaced nonwoven with ascorbic acid, produces 99.99 percent antibacterial activity.

TABLE 9
Description of Fabrics

| Fabric Name and Description | Fabric Treatment |
|---|---|
| TACGauze (TGz) | None |
| BIOGauze (BGz) | Pilot scale pad-dry Ascorbic acid |
| | on TACGauze |
| TACGauze, crosslinked | Pad-dry-cure of citric and ascorbic |
| (TGzX1) | acid (crosslinking) |
| TACGauze, sprayed (TGzS1) | Spray-dried 10 mM Ascorbic Acid |
| TACGauze, sprayed (TGzS2) | Spray-dried 10:1 mM ascorbic acid |
| | and copper(II) chloride (CuCl₂) |
| TACGauze, lab (TGzL) | Pad-dry of 10 mM Ascorbic acid |
| | (lab scale) |
| TACGauze, lab-pilot (TGzPL) | Pad-dry of 10 mM Ascorbic |
| | acid (lab-pilot scale using |
| | padding strength of 5-30 psi) |
| Plain weave woven 100% | Spray-dried lOmM Ascorbic Acid |
| cotton fabric, sprayed (GCS1) | |
| Plain weave woven 100% | Spray-dried with 10:1 mM ascorbic |
| cotton fabric, sprayed (GCS2) | acid and copper(II) chloride (CuCl₂) |
| 100% Bleached cotton | None |
| nonwoven (B8-s2) | |
| 100% cotton Fine mesh gauze | Bleached 100% cotton gauze |
| crosslinked (FMGzX1) | Pad-dry-cured with citric and |
| | ascorbic acid (crosslinking) |

TABLE 10
Antimicrobial Results using AATCC 100 Test Method

| | K. Pneumonia | S. aureus |
|---|---|---|
| | (4352) | (6538) |
| Sample Description | % Reduction after 24 hrs | |
| (TGz) | 0 | 0 |
| (BGz) | 99.99 | 99.99 |
| (TGzS1) | 99.9 | 99.9 |
| (TGzS2) | 99.9 | 99.9 |
| (TGzL) | 99.99 | 99.99 |
| (TGzPL) 5 psi | 99.99 | 99.99 |
| (TGzPL) 30 psi | 99.35 | 99.99 |
| (GCS1) | 0 | 0 |
| (GCS2) | 0 | 94.8 |
| (FMGzX1) | 99.99 | 99.99 |

### Example 7

### Hydrogen Peroxide Production

This Example was performed to determine whether the commercially based hemostatic fabric, TACGauze, could be modified by traditional finishing chemistry treatments to create a fabric that retained its hemostatic function but also possessed significant antibacterial and antiviral properties.

Hydrogen peroxide produced by the treated samples was determined using the ferrous oxidation with xylenol orange (FOX1) assay method 1 (Gay, C. and Gebicki, J. M., 2000, “A Critical Evaluation of the Effect of Sorbitol on the Ferric-Xylenol Orange Hydroperoxide Assay,” Anal. Biochem. 284 (2): 217-220; Wolff, S. P., 1994, “Ferrous ion oxidation in presence of ferric ion indicator xylenol orange for measurement of hydroperoxides,” In Methods in Enzymology, Academic Press, Vol. 233, pp 182-189). Briefly, stock solutions were made of the reagents to make the substrate solution to add to the sample aliquot to measure peroxide production spectrophotometrically. Approximately 50 mg of sample swatch was incubated overnight at room temperature in 2.0 mL of deionized water. Aliquots, 50 μL, in quadruplicate of the resulting sample swatch solution were used for the assay. The substrate solution consisted of 25 mM sulfuric acid (H₂SO₄), 100 μM xylenol orange, 100 mM sorbitol, and 250 μM ferrous ammonium sulphate final concentration in water. The sample aliquot, 50 μL, was added to 950 μL of substrate solution and shaken. The absorbance was measured at 560 nm at one-minute intervals for six minutes. A standard curve of hydrogen peroxide (H₂O₂), 0-10 μM concentration, was used to calculate the unknown peroxide content in test samples.

The results of the antimicrobial activity of the fabrics led to an effort to delineate the mechanism of action of multiple ascorbic acid-based dressing formularies that could potentially be used as barrier fabrics for antimicrobial and antiviral activity.

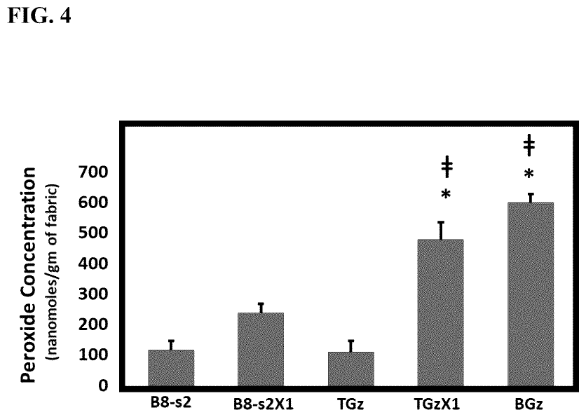

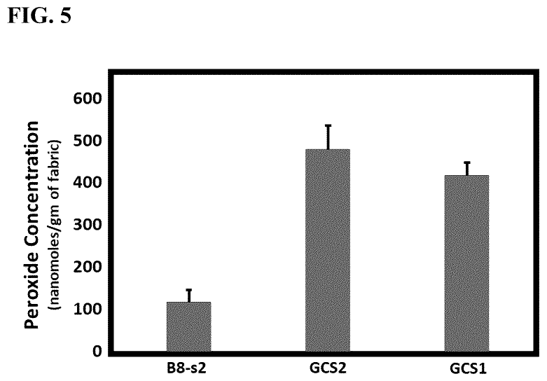

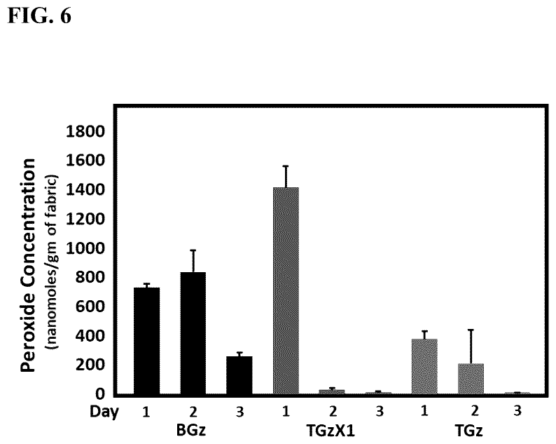

The data shown in FIG. 4 to FIG. 6 demonstrate the hydrogen peroxide levels generated by the functional fabrics of this study. In this regard it is interesting that the levels of hydrogen peroxide produced per gram of fabric is consistent with the antibacterial results shown in Table 10. As shown in FIG. 4 and FIG. 5, levels of hydrogen peroxide that correlate with the antibacterial activity were observed to be 250 to 550 nanomoles per gram of fabric. When assessed for duration of hydrogen peroxide activity (FIG. 6), the levels were also commensurate with observed antibacterial activity present after three days in BGz. This figure also shows that in formulation TGzX1 that consisted of ascorbic acid crosslinked to cellulose levels were robust at day one but fall off thereafter, most likely due to differences in finishing conditions.

### Example 8

### Antiviral Activity

The treated cotton blend nonwoven BIOGauze (BGz) was tested for antiviral activity using TGz as a control to test for efficacy against a challenge virus and one that holds high homology with the Severe Acute Respiratory Syndrome Coronavirus 2 (SARS-COV-2) virus (88%).

One of the routine challenge assays to screen for any antiviral activity regarding textiles is the AATCC 100 test method (TM), “Assessment of Antibacterial Finishes on Textile Materials” modified for viruses using the MS2 bacteriophage (MS2). MS2 is a non-enveloped positive-stranded RNA virus of the bacteriophage family Leviviridae. Its small size, icosahedral structure, and environmental resistance have made MS2 ideal for use as a surrogate virus in disinfectant studies. The permissive host cell for MS2 is Escherichia coli. The study showed that BGz reduced viral load by 99.99 percent after one hour compared with the control and TGz, BGz precursor, at time zero (Table 11). Notably as well the Human coronavirus, Strain 229E (HCoV-229E) was also used in the AATCC 100 TM modified for viruses assay. The cell host, in this case, is MRC-5 (ATCC CCL-171) from the human lung fibroblast cell line. A 90% reduction with BGz and no reduction in TGz or commercial N-95 mask sample at a time point of six hours (see Table 12).

AATCC100 test method for antibacterial finishes on textile materials modified for viruses was used to test for antiviral activity using the surrogate virus, MS2 Bacteriophage ATCC 15597-B1 (performed by Microchem Laboratory). The limit of detection for this assay is 1.00E+01 PFU/carrier and is reported as <1.00E+01 PFU/carrier in the table below.

TABLE 11
Preliminary antiviral screening results

| | Contact | Average | % | Logio |
|---|---|---|---|---|
| Carrier | Time | PFU/Carrier^{b} | Reduction | Reduction |
| Control | 5 minutes | 1.78E + 07 | | |
| (Time zero) | 1 hour | 1.47E + 06 | N/A | N/A |
| | 3 hours | 1.47E + 06 | | |
| Control | 5 minutes | 2.25E + 07 | | |
| | 1 hour | 1.50E + 06 | N/A | N/A |
| | 3 hours | 2.10E + 06 | | |
| TACGauze | 5 minutes | 3.63 + 06 | 79.55 | 0.69 |
| | 1 hour | 9.67E + 05 | 34.09 | 0.18 |
| | 3 hours | 1.83E + 06 | No Reduction | No Reduction |
| BIOGauze | 5 minutes | 7.00 + 06 | 60.60 | 0.40 |
| | 1 hour | 2.13E + 02 | 99.99 | 3.84 |
| | 3 hours | 1.47E + 06 | >99.99932 | >5.17 |

AATCC100 test method for antibacterial finishes on textile materials modified for viruses was used to test for antiviral activity using Human coronavirus, strain 229E, ATCC VR-740; host cell MRC-5 (ATCC CCL-171) (performed by Microchem Laboratory). TCID₅₀ (Tissue Culture Infectivity Dose) represents the endpoint dilution where 50% of the host cell monolayers exhibit cytotoxicity, determined using Spearman-Kärber method.

TABLE 12
Preliminary results of Virucidal activity

| | TCID₅₀ per | % | Log |
|---|---|---|---|
| | 0.1 mL^{b} | Reduction | Reduction |
| Virus titer | 5.50 log₁₀ | | |
| Virus (time zero) | 5.50 log₁₀ | | |
| Virus control | 3.50 log₁₀ | | |
| TACGauze | 4.50 log₁₀ | No reduction | No reduction |
| BIOGauze | 2.50 log₁₀ | 90% | 1.00 |
| N95 Mask material | 5.00 log₁₀ | No reduction | No reduction |

The data presented in this Example shows that BIOGauze is capable of reducing Human coronavirus, strain 229E, while TACGauze, and N95 mask material presented no reduction.

### Example 9

### Ascorbic Acid Crosslinking to Cellulose in Cotton Fabrics

As seen in the examples above, the formulation of ascorbic acid in cotton nonwovens gave rise to robust antimicrobial and antiviral levels of hydrogen peroxide. Thus, it was of interest to demonstrate that a covalently attached form of ascorbic acid and cellulose e.g., an ascorbate-cellulose conjugate elicits comparable hydrogen peroxide activity. To do this crosslinking of ascorbic acid to cotton cellulose in similar fabrics as those described here was attempted.

FIG. 1 shows the chemistry of covalently attaching ascorbic acid to the cellulose in cotton by way of a polycarboxylic acid. The polycarboxylic acid employed in this study was citric acid. It is important to note that polycarboxylic acid approaches to crosslinking cellulose in cotton have previously been extensively studied in the context of creating durable press fabric (Andrews, B. A., 1990, “Nonformaldehyde DP finishing of cotton with citric acid,” Text. Chem. Color. 22:63-67; Caulifield, D. F., 1994, “Ester crosslinking to improve wet performance of paper using multifunctional carboxylic acids, butanetetracarboxylic and citric acid,” TAPPI J. 77:205-212; Welch, C. M. and Andrews, B. K., 1989, “Catalysts and processes for formaldehyde-free durable press finishing of cotton textiles with polycarboxylic acids, U.S. Pat. No. 4,820,307). This work underscores the importance of acidic conditions or even an acid catalyst such as sodium hypophosphite (SHP) (Welch, C. M. and Andrews, B. A. K., 1989, “Ester crosslinks: a route to high performance nonformaldehyde finishing of cotton,” Text. Chem. Color. 21:13-17. As shown in FIG. 1, crosslinking of ascorbic acid to the cellulose in cotton proceeds through the acid-catalyzed formation of an acid anhydride. FIG. 1 shows a schematic of a Crosslinking Reaction of Ascorbic Acid Linked to Cellulose. Ascorbic acid [(R)-5-[(S)-1,2,-dihydroxyethyl]-3,4-dihydroxyfuran-2 (5H)-one] is reacted with citric acid under conventional pad dry cure conditions. The reaction is shown at the 1-hydroxy position of ascorbic acid to form the ester crosslinked analog of cellulose.

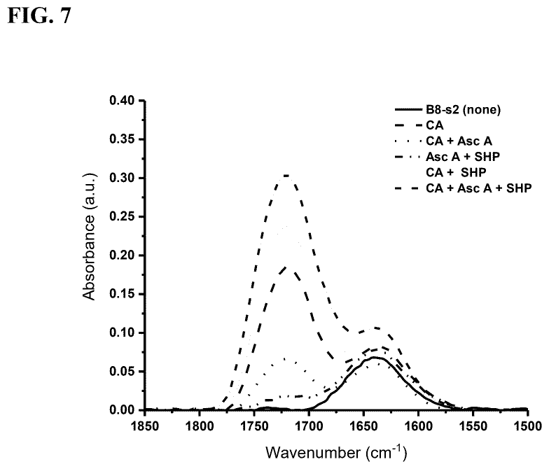

The IR region displayed in FIG. 7 was utilized to characterize fabrics subject to this treatment. It is notable that wide bands ranging between 1675 cm⁻¹ and 1800 cm⁻¹ appeared for the treated fabrics of this study, while this same feature is missing for the untreated sample (B8-s2 (none)). This range, 1675 cm⁻¹ and 1800 cm⁻¹, coincides with the expected region of ester C═O groups (Lyu, W. and Wehby, G. L., 2020, “Community Use Of Face Masks And COVID-19: Evidence From A Natural Experiment Of State Mandates In The US,” Health Aff (Millwood), 39 (8), 1419-1425). For the fabric treated with citric acid, a moderately intense band appeared with a peak point at 1721 cm⁻¹ (CA). However, treatment of a bleached nonwoven fabric with a combination of the citric acid and ascorbic acid produced a band at 1724 cm⁻¹ (CA+Asc A). Treatment with citric acid and the SHP catalyst increased the intensity of the band, and the peak point was seen at 1721 cm⁻¹ (CA+SHP). Moreover, the highest intensity band in the ester region was observed for the fabric treated with the combination of citric acid, ascorbic acid, and SHP, with a peak point at 1723 cm⁻¹ (CA+Asc A+SHP). This result is also correlated with the highest add-on. Fabrics treated with ascorbic acid displayed low-intensity ester bands at 1725 cm⁻¹, and those treated with SHP displayed low-intensity ester bands at 1723 cm⁻¹. Remarkable in this study is that the intensity of the ester bands followed the percent add-ons observed for the fabrics, with the ascorbic acid-treated fabric showing the lowest intensity band and add-on (1.56%), and the fabric treated with citric acid, ascorbic acid, and SHP, showing the highest intensity and add-on (9.43%). Thus, given the thoroughly washed fabrics do not retain unreacted reagents, the correlation of IR bands to increased add-ons strongly suggests the formation of a covalent linkage between ascorbic acid and cellulose.

As described above, small differences in band peak positions were observed for the various treatments. Fabrics treated with citric acid, or the combination of citric acid and SHP presented a band peak centered at 1721 cm⁻¹. The similarity in these bands suggests that the addition of the SHP does not change the nature of the citrate cross-linkage but does increase its formation. In contrast, the addition of ascorbic acid to the citric acid and SHP treatment mixture slightly shifted the position of the ester band to 1723 cm⁻¹. Shifts in the band peak position to 1723 cm⁻¹ for all fabrics treated with ascorbic acid, further supports covalent linkage of ascorbic.

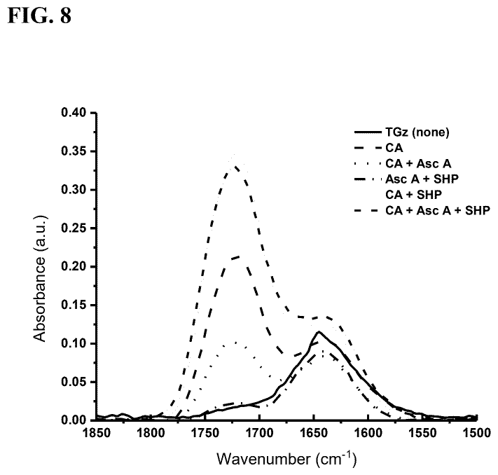

As seen in FIG. 8, treated TGz gauze fabrics showed similar FTIR changes. A notable deviation for the TGz fabrics is the observed intensity of ester bond for the citric acid and SHP-treated fabric exceeding the intensity of the fabric treated with citric acid, ascorbic acid, and SHP. However, the close proximity of add-on values for these two fabrics (6.82% for the former, 7.16% for the latter) likely accounts for the discrepancy.

### Example 10

Antibacterial and H₂O₂ Activity of Cotton/Hemp Fabrics

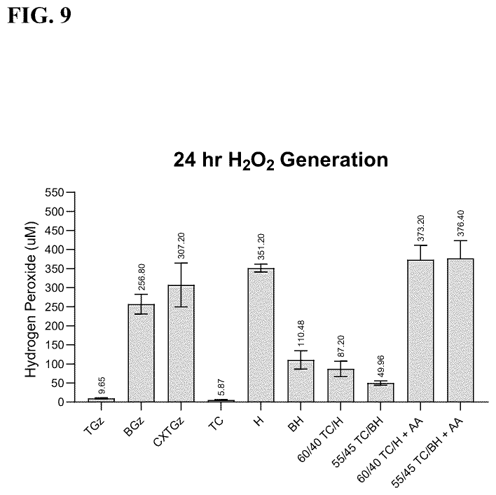

FIG. 9 depicts a graph of the hydrogen peroxide produced by fabrics treated in different ways. The Y Axis presents the hydrogen peroxide level (micromolar, μM) of fabric. The X Axis presents the different fabrics: TACGauze (TGz); BIOGauze (BGz); citrate crosslinked TACGauze (CXTGz); TruCotton (TC); unbleached hemp (H), bleached hemp (BH); 60% TruCotton/40% unbleached hemp (60/40 TC/H); 55% TruCotton/45% bleached hemp (55/45 TC/BH); 60% TruCotton/40% unbleached hemp treated with ascorbic acid (60/40 TC/H+AA); and 55% TruCotton/45% bleached hemp treated with ascorbic acid (55/45 TC/BH+AA). Hydrogen peroxide content was determined using the Amplex Red Assay which is performed fluorometrically utilizing Amplex Red reagent (10-acetyl-3,7-dihydroxyphenoxazine) in combination with horseradish peroxidase (HRP), n=4. Error bars represent Standard Error of Mean (SEM). Results were analyzed by ANOVA. Post hoc (Tukey) demonstrated significantly higher levels of H₂O₂ production by 60% TruCotton/40% unbleached hemp treated with ascorbic acid (60/40 TC/H+AA) and 55% TruCotton/45% bleached hemp treated with ascorbic acid (55/45 TC/BH+AA) versus TruCotton (TC) alone, unbleached hemp (H) alone, bleached hemp (BH) alone, 60% TruCotton/40% unbleached hemp (60/40 TC/H) alone, and 55% TruCotton/45% bleached hemp (55/45 TC/BH) alone.

Using the AATCC 100 test method, the antibacterial results (Table 13) for these fabrics against both gram-positive and gram-negative bacteria generally correspond to their respective H₂O₂ production. For this analysis, Gram-positive Staphylococcus aureus American Type Culture Collection (ATCC) 6538 (S. aureus) and Gram-negative Klebsiella pneumoniae ATCC 4352 (K. pneumoniae) were selected for use in this assay because they are the standard microorganisms for the method and they represent organisms that are commonly found to colonize and infect wounds. The 60% TruCotton/40% unbleached hemp treated with ascorbic acid (60/40 TC/H+AA) and the 55% TruCotton/45% bleached hemp treated with ascorbic acid (55/45 TC/BH+AA) demonstrated superior antimicrobial results against both gram-positive and gram-negative bacteria as compared to the 60% TruCotton/40% unbleached hemp (60/40 TC/H) alone and the 55% TruCotton/45% bleached hemp (55/45 TC/BH) alone.

The compositions for the ten different fabrics shown in FIG. 9 are listed below:
1: TACGauze (TGz):
   60% TruCotton fibers, 20% scoured/bleached cotton fibers, 20% polypropylene fibers.
2: BIOGauze (BGz):
   60% TruCotton fibers, 20% scoured/bleached cotton fibers, 20% polypropylene fibers. Fabrics were treated with 1% (w/v) L-ascorbic acid with 0.6% (w/v) 1-hexanol as a wetting agent in ultrapure water using a pad-dry-cure method.
3: Citrate crosslinked TACGauze (CXTGz):
   60% TruCotton fibers, 20% scoured/bleached cotton fibers, 20% polypropylene fibers. Fabrics were treated with 7% (w/v) citric acid, 5% (w/v) L-ascorbic acid, 4% (w/v) sodium hypophosphite with 0.6% (w/v) 1-hexanol as a wetting agent in ultrapure water using a pad-dry-cure method.
4: TruCotton (TC):
   100% greige cotton fibers that have undergone a proprietary cleaning process to remove all visible foreign matter (VFM).
5: Hemp (H):
   100% commercially available raw hemp fibers.
6: Bleached hemp (BH):
   100% commercially available bleached hemp fibers.
7: 60% TruCotton, 40% unbleached hemp (60/40 TC/H):
   blends of compositions 4, and 5., above.
8: 55% TruCotton, 45% bleached hemp (55/45 TC/BH):
   blends of compositions 4, and 6., above.
9: 60% TruCotton, 40% unbleached hemp treated with ascorbic acid (60/40 TC/H+AA):
   blends of compositions 4, and 5., above, treated with 1% (w/v) L-ascorbic acid with 0.6% (w/v) 1-hexanol as a wetting agent in ultrapure water using a pad-dry-cure method. 10: 55% TruCotton, 45% bleached hemp treated with ascorbic acid (55/45 TC/BH+AA): blends of compositions 4, and 6., above, treated with 1% (w/v) L-ascorbic acid with 0.6% (w/v) 1-hexanol as a wetting agent in ultrapure water using a pad-dry-cure method.

TABLE 13
Antibacterial Results and H₂O₂ Generation of Cotton/Hemp Fabrics

| | Per cent antibacterial in | Hydrogen |
|---|---|---|
| | AATCC 100 Assay | Peroxide Levels |
| Fabric | Gram −/ Gram + | (micromolar) |
| 60/40 TC/H | 98.61/97.12 | 87.20 |
| 55/45 TC/BH | 99.36/94.80 | 49.96 |
| 60/40 TC/H + AA | 99.86/99.98 | 372.20 |
| 55/45 TC/BH + AA | >99.99/99.89 | 376.40 |

We claim:

1. A single layered nonwoven fabric comprising about 5% by weight to about 95% by weight non-scoured, non-bleached greige cotton fibers, about 0% by weight to about 95% by weight bleached cotton fibers, and about 5% by weight to about 95% by weight hemp fibers, all weight percentages adding up to 100%.

2. The single layered nonwoven fabric according to claim 1, wherein the fabric comprises about 60% by weight non-scoured, non-bleached greige cotton fibers, about 20% by weight bleached cotton fibers, and about 20% by weight hemp fibers.

3. The single layered nonwoven fabric according to claim 1, wherein the non-scoured, non-bleached greige cotton has a purity level of about 99.9%.

4. The single layered nonwoven fabric of claim 1, wherein the fabric has a density higher than about 30 g/m².

5. The single layered nonwoven fabric of claim 1, wherein the fabric has an increased absorption capacity of about 60% to about 70% when compared with a fabric not comprising non-scoured, non-bleached greige cotton fibers.

6. The single layered nonwoven fabric of claim 1, further comprising ascorbic acid or sodium ascorbate.

7. The single layered nonwoven fabric of claim 6, wherein the fabric comprises about 10 mM ascorbic acid or sodium ascorbate.

8. The single layered nonwoven fabric of claim 1, further comprising ascorbic acid, citric acid, sodium hypophosphite, or a mixture thereof.

9. The single layered nonwoven fabric of claim 1, wherein the fabric comprises about 60% by weight non-scoured, non-bleached greige cotton fibers, and about 40% by weight unbleached hemp.

10. The single layered nonwoven fabric of claim 1, wherein the fabric comprises about 55% by weight non-scoured, non-bleached greige cotton fibers, and about 45% by weight bleached hemp.

11. The single layered nonwoven fabric of claim 1, wherein the fabric comprises about 60% by weight non-scoured, non-bleached greige cotton fibers, and about 40% by weight unbleached hemp, wherein the fabric has been treated with ascorbic acid.

12. The single layered nonwoven fabric of claim 1, wherein the fabric comprises about 55% by weight non-scoured, non-bleached greige cotton fibers, and about 45% by weight bleached hemp, wherein the fabric has been treated with ascorbic acid.

13. The single layered nonwoven fabric of claim 11, wherein the fabrics were treated with 7% (w/v) citric acid, 5% (w/v) L-ascorbic acid, and 4% (w/v) sodium hypophosphite with 0.6% (w/v) 1-hexanol as a wetting agent in ultrapure water using a pad-dry-cure method.

14. The single layered nonwoven fabric of claim 12, wherein the fabrics were treated with 7% (w/v) citric acid, 5% (w/v) L-ascorbic acid, and 4% (w/v) sodium hypophosphite with 0.6% (w/v) 1-hexanol as a wetting agent in ultrapure water using a pad-dry-cure method.

15. The single layered nonwoven fabric of claim 11, wherein the fabrics were treated with 1% (w/v) L-ascorbic acid with 0.6% (w/v) 1-hexanol as a wetting agent in ultrapure water using a pad-dry-cure method.

16. The single layered nonwoven fabric of claim 12, wherein the fabrics were treated with 1% (w/v) L-ascorbic acid with 0.6% (w/v) 1-hexanol as a wetting agent in ultrapure water using a pad-dry-cure method.

17. The single layered nonwoven fabric of claim 6, wherein the ascorbic acid is covalently attached to at least one cellulosic portion of the fabric composition.

18. An article of manufacture prepared with the fabric composition of claim 1.

19. The article of manufacture of claim 18, wherein the article of manufacture is a protective textile.

20. The article of manufacture of claim 19, wherein the protective textile is a surgical arena fabric, a surgical personnel protective garment, a wound dressing, a non-wound patient dressing, a bandage, a gauze, a packing, a mask, or a cleaning material.

21. The single layered nonwoven fabric according to claim 1, wherein the nonwoven fabric is produced by a process comprising:
(a) preparing needle punched webs of the fibers,
(b) uniformly hydroentangling the webs using a system wherein the system is equipped with one low water pressure jet head that wets the webs on the top face of the webs to form a wetted substrate and wherein the system is equipped with two high water pressure jet heads that subsequently alternatively wets the wetted substrate on either face of the wetted substrate, wherein the system utilizes an about 23 mesh to about 17 mesh screen, and
(c) drying the wetted substrate to form the fabric.

22. A method for preparing the fabric composition of claim 1, wherein the method comprises:
saturating a fabric composition with citric acid, ascorbic acid, sodium hypophosphite, or a mixture thereof,
padding the saturated fabric composition,
drying the padded fabric composition at a first temperature, and
curing the dried fabric composition at a second temperature higher than the first temperature.

23. The method of claim 22, wherein the ascorbic acid is covalently attached to at least one cellulosic portion of the fabric composition.

24. The method of claim 22, wherein the fabric composition is a cloth, a woven fabric, a knitted fabric, a nonwoven fabric, or a final article.

25. The method of claim 22, wherein the fabric composition is a nonwoven fabric.

26. The method of claim 25, wherein the nonwoven fabric is a single layered fabric or a multilayered fabric.

27. The method of claim 22, wherein the fabric composition is a woven fabric.

28. A multi-layered nonwoven wound dressing, comprising at least one inner layer containing about 50% by weight to about 95% by weight non-scoured, non-bleached greige cotton fibers and about 5% by weight to about 50% by weight hemp fibers, all weight percentages adding up to 100%, and at least one outer layer containing about 5% by weight to about 95% by weight non-scoured, non-bleached greige cotton fibers, about 0% by weight to about 95% by weight bleached cotton fibers, and about 5% by weight to about 60% by weight hemp fibers, all weight percentages adding up to 100%.
